(19) United States
(12) Patent Application Publication
(10) Pub. No.: US 2021/0369164 A1
(43) Pub. Date: Dec. 2, 2021

(54) SENSOR ASSEMBLY WITH MOVABLE SKIN SENSOR

(71) Applicant: MediBeacon Inc., St. Louis, MO (US)

(72) Inventor: Jim Harr, St. Louis, MO (US)

(21) Appl. No.: 17/331,341

(22) Filed: May 26, 2021

Related U.S. Application Data
(60) Provisional application No. 63/029,927, filed on May 26, 2020.

(51) Int. Cl. A61B 5/20; A61B 5/00; A61M 1/36

(52) U.S. Cl. CPC A61B 5/201; A61B 5/6833; A61B 2560/0412; A61B 5/0071; A61B 2562/146; A61M 1/3639

(57) ABSTRACT

Disclosed herein is a housing assembly configured to attach to a body surface of a patient and comprising at least one opening; a skin sensor movably disposed in the at least one opening of the housing assembly; and wherein the housing assembly is configured to move independently relative to the skin sensor when an external force is applied to the housing assembly.

### FIELD

The field of the disclosure relates generally to sensor systems. In particular, this disclosure generally relates to a sensor assembly wherein the skin sensor is movably attached to the attachment collar and/or cover and configured to move independently of the attachment collar and/or cover when an external force is applied to the sensor assembly.

### BACKGROUND

In the clinical and preclinical field, determining various organ functions is accorded great importance since, for example, corresponding therapies or medications can be controlled in accordance with said organ functions. The sensor assembly is described hereinafter substantially with regard to kidney function monitoring. In principle, however, other applications are also conceivable in which the function of a particular organ can be detected by means of determining a temporal profile of an indicator substance. Other applications may include gastrointestinal (GI) monitoring, Continuous Renal Replacement Therapy (CRRT) therapy, blood-brain barrier monitoring, and the like.

The glomerular filtration rate (GFR) is an important clinical parameter to assess the level of kidney function in a patient. As shown in the table below, the lower the GFR, the more serious the kidney impairment for Chronic Kidney Disease (CKD) and other renal insufficiencies. The GFR can be estimated based on a blood test measuring the blood creatinine level in the patient in combination with other factors. More accurate methods involve the injection of an exogenous substance into a patient followed by careful monitoring of plasma and/or urine concentration over a period of time. These are often contrast agents (CA) that can cause renal problems on their own. Radioisotopes or iodinated aromatic rings are two common categories of CAs that are used for GFR determination.

| Stage | Description | GFR* |
|---|---|---|
| Increased | Increase of risk factors (e.g., diabetes, high | >90 |
| risk | blood pressure, family history, age, ethnicity) | |
| 1 | Kidney damage with normal kidney function | >90 |
| 2 | Kidney damage with mild loss of kidney | 60-89 |
| | function | |
| 3a | Mild to moderate loss of kidney function | 44-59 |
| 3b | Moderate to severe loss of kidney function | 30-44 |
| 4 | Severe loss of kidney function | 15-29 |
| 5 | Kidney failure; dialysis required | <15 |

*GFR is measured in units of mL/min/1.73 m².

With regard to conventional renal function measurement procedures, an approximation of a patient's GFR can be made via a 24 hour urine collection procedure that (as the name suggests) typically requires about 24 hours for urine collection, several more hours for analysis, and a meticulous bedside collection technique. Unfortunately, patient compliance using this method is very low, and, as a consequence, is not generally utilized by clinicians.

Examples of exogenous substances capable of clearing the kidney exclusively via glomerular filtration (hereinafter referred to as “GFR agents”) include creatinine, o-iodohippuran, and ^{99m}Tc-DTPA. Examples of exogenous substances that are capable of undergoing renal clearance via tubular secretion include ⁹⁹mTc-MAG3 and other substances known in the art. ⁹⁹mTc-MAG3 is also widely used to assess renal function though gamma scintigraphy as well as through renal blood flow measurement. One drawback to many indicator substances, such as o-iodohippuran, ⁹⁹mTc-DTPA and ⁹⁹mTc-MAG3, is that they are radioisotopes and therefore require special handling techniques and are associated with risks to patient health.

### BRIEF SUMMARY

Disclosed herein is a sensor assembly. The sensor assembly generally comprises: a housing assembly configured to attach to a body surface of a patient and comprising at least one opening; and a skin sensor movably disposed in the at least one opening of the housing assembly; wherein the housing assembly is configured to move independently relative to the skin sensor when an external force is applied to the housing assembly.

In another aspect, disclosed herein is a sensor assembly, wherein the sensor assembly generally comprises: an attachment collar configured to attach to a body surface of a patient and comprising at least one opening; a skin sensor movably disposed in the at least one opening of the attachment collar; and a cover attached to the attachment collar; wherein the attachment collar and cover are configured to move independently relative to the skin sensor when an external force is applied to the sensor assembly.

In another aspect, disclosed herein is a sensor assembly, wherein the sensor assembly generally comprises: an attachment collar configured to attach to a body surface of a patient and comprising at least one opening, a skin sensor disposed in the at least one opening of the attachment collar, and a cover attached to the attachment collar and at least partially enclosing the skin sensor; wherein the cover is configured to rotate independently of the skin sensor when a rotational force is applied to the sensor assembly.

### BRIEF DESCRIPTION OF THE DRAWINGS

FIG. 1 illustrates one embodiment of the sensor assembly that includes a locking bar on each side of the sensor to secure it to the attachment collar, a cord management system to provide strain relief and security from cord pulls, and a pull tab for easy removal of the attachment collar from skin.

FIG. 2 illustrates one embodiment of the sensor assembly for attaching the skin sensor to the attachment collar using selectively adhesive surfaces.

FIG. 3 illustrates one embodiment of the sensor that includes a bar code/QR reader.

FIG. 4 illustrates a torturous light path for the connection between the skin sensor and the attachment collar to ensure a light-tight fit.

FIG. 5 illustrates a cam lock between the skin sensor and the attachment collar.

FIG. 6 illustrates one embodiment of the sensor assembly that comprises tabs to aid the alignment and positioning of the skin sensor relative to the attachment collar.

FIG. 7 illustrates a stretch pocket attachment collar for the sensor assembly that provides a slight downward pressure to the skin sensor onto the skin of the patient.

FIG. 8 illustrates one embodiment of the sensor assembly that includes RFID authentication and grooves within the attachment collar for the cord to provide security from cord pulls.

FIG. 9 illustrates one embodiment of the sensor assembly that includes a lock and key type security feature between the attachment collar and the skin sensor.

FIG. 10 illustrates one embodiment of the sensor assembly that includes a magnetic connection between the attachment collar and the skin sensor.

FIG. 11 illustrates one embodiment of the sensor assembly that includes an attachment collar that encircles the sensor, and a cable management system to provide strain relief and security from cord pulls.

FIG. 12 illustrates one embodiment of the sensor assembly that includes an embedded chemical in the attachment collar that can be detected by the sensor.

FIG. 13 illustrates one embodiment of the sensor assembly that includes a swivel attachment between the skin sensor and the attachment collar.

FIG. 14 illustrates one embodiment of the sensor assembly that includes a tab placement port and cord management system to provide strain relief and security from cord pulls.

FIG. 15 illustrates one embodiment of the sensor assembly that includes fold-over tabs to secure the skin sensor to the attachment collar.

FIG. 16 illustrates one embodiment of the sensor assembly that includes a communication port (e.g., an EEPROM) between the skin sensor and the attachment collar.

FIG. 17 illustrates one embodiment of the sensor assembly that includes a cam lock to secure the skin sensor to the attachment collar, and providing a slight downward pressure to the skin sensor onto the skin of the patient and visual indication that the sensor is locked in place.

FIG. 18 illustrates one embodiment of the sensor assembly that includes a wrap mechanism to secure the skin sensor to the attachment collar, and providing a slight downward pressure to ensure a light-tight fit.

FIG. 19 illustrates one embodiment of the sensor assembly that includes clips on the skin sensor which secure the sensor to the attachment collar, a cord management system to provide strain relief and security from cord pulls, and a pull tab for easy removal of the attachment collar from the skin when the session is complete.

FIG. 20 illustrates one embodiment of the sensor assembly that includes a locking mechanism on the skin sensor which secures the sensor to the attachment collar, a cord management system to provide strain relief and security from cord pulls, and a pull tab for easy removal of the attachment collar from the skin when the session is complete.

FIG. 21 illustrates one embodiment of the sensor assembly that includes an attachment collar that encircles the sensor to ensure a light-tight fit, a cord management system to provide strain relief and security from cord pulls, and a pull tab for easy removal of the attachment collar from the skin when the session is complete.

FIG. 22 illustrates one embodiment of the sensor assembly that includes clips on the skin sensor which secure the sensor to the attachment collar, a cord management system to provide strain relief and security from cord pulls, and a pull tab for easy removal of the attachment collar from the skin when the session is complete.

FIG. 23A illustrates an exploded perspective view of one embodiment of the sensor assembly including a movable skin sensor.

FIG. 23B illustrates a perspective view of one embodiment of an assembled sensor assembly including a movable skin sensor.

FIG. 24A illustrates a perspective view of an embodiment of a movable skin sensor.

FIG. 24B illustrates an exploded perspective view of an embodiment of a movable skin sensor disposed between a cover and a frame.

FIGS. 24C-D illustrate cross-sectional front and side views, respectively, of additional degrees of freedom of movement imparted by the features in FIGS. 24A-B.

FIG. 25 illustrates a cross-sectional side view of an embodiment of the sensor assembly including a movable skin sensor configured to slide in an upward and downward direction independently of the cover and/or attachment collar when a downward force is applied to the cover and/or attachment collar.

FIG. 26 illustrates a cross-sectional side view of an embodiment of the sensor assembly including a movable skin sensor with a spring configured to prevent the transfer of force to the movable skin sensor when an external force is applied to the cover and/or attachment collar.

FIGS. 27A-B illustrate a perspective bottom view of an embodiment of a cover of the sensor assembly and further illustrate the positioning of springs in embodiments including a movable skin sensor.

FIGS. 28A-B illustrate a top and side view of an embodiment of a movable skin sensor including an alternative spring assembly including leaf springs.

FIGS. 29A-B illustrate a top and side view of an embodiment of a movable skin sensor including an alternative spring assembly including a plurality of springs positioned around a perimeter of the movable skin sensor.

FIG. 30A illustrates a side view of an embodiment of a movable skin sensor including a spring assembly integrally molded to the cover of the sensor assembly.

FIG. 30B illustrates a side view of an embodiment of a movable skin sensor including a spring assembly integrally molded to the sliding chassis or frame of the sensor assembly

FIGS. 31A-B illustrate a perspective and side view of an alternative embodiment of the sensor assembly including a rotatable circular top cover, rotatable cable and rotatable movable skin sensor.

FIG. 31C illustrates a side view of a rotatable movable skin sensor that is configured to rotate independently relative to the attachment collar.

FIG. 31D illustrates a cross-sectional side view of an alternative embodiment of the sensor assembly including a rotatable movable skin sensor with a spring configured to prevent the transfer of force to the movable skin sensor when an external force is applied to the cover and/or attachment collar.

Unless otherwise indicated, the drawings and figures provided herein illustrate features of embodiments of the disclosure or results of representative experiments illustrating some aspects of the subject matter disclosed herein. These features and/or results are believed to be applicable in a wide variety of systems including one or more embodiments of the disclosure. As such, the drawings are not intended to include all additional features known by those of ordinary skill in the art to be required for the practice of the embodiments, nor are they intended to be limiting as to possible uses of the methods disclosed herein.

### DETAILED DESCRIPTION

In the following specification and the claims, reference will be made to a number of terms, which shall be defined to have the following meanings. The singular forms “a,” “an,” and “the” include plural references unless the context clearly dictates otherwise. The terms “comprising,” “including,” and “having” are intended to be inclusive and mean that there may be additional elements other than the listed elements. “Optional” or “optionally” means that the subsequently described event or a circumstance may or may not occur, and that the description includes instances where the event occurs and instances where it does not.

Approximating language, as used herein throughout the specification and claims, may be applied to modify any quantitative representation that could permissibly vary without resulting in a change in the basic function to which it is related. Accordingly, a value modified by a term or terms, such as “about,” “approximately,” and “substantially,” are not to be limited to the precise value specified. In at least some instances, the approximating language may correspond to the precision of an instrument for measuring the value. Here and throughout the specification and claims, range limitations may be combined and/or interchanged; such ranges are identified and include all the sub-ranges contained therein unless context or language indicates otherwise.

As used herein, the term “light-tight” means the interface between two surfaces does not permit the passage of external light. For example, when the attachment collar is placed on a body surface and the skin sensor is operably attached to it, a surface of the skin sensor faces the body surface. No external light penetrates to the body surface between the interface of the skin sensor and the attachment collar to reach the area of the body surface that faces the skin sensor. Additionally, no external light passes between the body surface of the patient and the surface or edges of the attachment collar adhered to or in contact with the body surface. As such, the only light detected by the skin sensor emanates directly incident to the body surface of the patient. In some aspects, the only light detected by the skin sensor emanates from a response light generated by an indicator substance inside the body of the patient.

As used herein, the term “movable” denotes the ability to move, slide, articulate, tilt or pivot in any direction along an x, y, z axis, as well as the ability to rotate in a clockwise and/or counterclockwise direction relative to, e.g., a housing assembly as described herein.

PCT/EP2009/060785, which is incorporated by reference herein in its entirety for all purposes, discloses skin sensors that can, in some aspects, be configured for use in conjunction with an attachment collar thereby creating a sensor assembly as disclosed herein.

The term “patient” as used herein refers to a warm blooded animal such as a mammal which is the subject of a medical treatment for a medical condition that causes at least one symptom. It is understood that at least humans, dogs, cats, and horses are within the scope of the meaning of the term. In some aspects, the patient is human. As used herein, any suitable surface on the body of the patient may be used as the body surface. Examples include, but are not limited to, skin surfaces, fingernails or toenails, more particularly surfaces exposed to the atmosphere. Generally, as used herein, the term “patient” means a human or an animal on which at least one of the sensor assembly may be used, independently of the health of the patient.

The skin sensor comprises at least one radiation source. A radiation source is understood to be any device which can emit radiation anywhere on the electromagnetic spectrum. In some aspects, the electromagnetic radiation is in the visible, infrared, ultraviolet, and/or gamma spectral range. Alternatively or additionally, other types of radiation can also be used, for example streams of particles. By way of example and not limitation, alpha rays and/or beta rays may be used. The radiation source is configured to generate radiation of the type mentioned. Without restricting the type of radiation used and for convenience only, hereinafter radiation is generally designated as “light” whether or not it is in the visible region of the electromagnetic spectrum, and the radiation source is described more particularly with reference to a “light source”. However, other configurations of the radiation source are possible, in some aspects, and it is also possible, in some aspects, to combine different types of radiation sources.

The radiation source can be, for example, an integral constituent of the skin sensor, for example in the context of a layer construction of the skin sensor. The radiation source is therefore designed to generate at least one interrogation light directly within the skin sensor, in contrast to external generation of the interrogation light. In this respect, the skin sensor differs, for example, from the fiber-optic construction in U.S. Pat. No. 6,995,019 B2, in which an external light source is used. Instead of an individual light source, in some aspects, it is also possible to use a plurality of light sources, for example redundant light sources for emitting one and the same wavelength, and/or a plurality of different light sources for emitting different wavelengths. Generally, the at least one light source is designed to irradiate the body surface with at least one interrogation light.

An interrogation light is understood to be a light that can be used for the detection of an indicator substance as disclosed elsewhere herein, whose light excites the indicator substance inside a body tissue and/or a body fluid of the patient, for example with variable penetration depth, and causing a perceptible response, more particularly, an optically perceptible response. This excitation takes place in such a way that a luminescence, a fluorescence and/or a phosphorescence is initiated in the indicator substance. In some aspects, other types of excitation occur, for example scattering of the light at an identical or shifted wavelength. Generally, at least one response light is generated by the indicator substance in response to the interrogation light.

The interrogation light is designed such that the desired response is excited in a targeted manner in the indicator substance. Accordingly, by way of example and not limitation, a wavelength and/or a wavelength range of the interrogation light and/or some other property of the interrogation light can be adapted or adjusted based on the identity and properties of the indicator substance. This can be done directly by the radiation source, for example, by virtue of the radiation source providing the interrogation light having a specific wavelength and/or in a specified wavelength range and/or by the inclusion of at least one excitation filter being used to filter out the desired interrogation light from a primary light of the light source. In some aspects, the skin sensor performs fluorescence measurements on the indicator substance. Accordingly, the interrogation light can be adapted to the excitation range of the fluorescence of the indicator sub stance.

The skin sensor further comprises at least one detector designed to detect at least one response light incident from the direction of the body surface. The response light can be light in the sense of the above definition. The detector is also an integral constituent of the skin sensor. The detector is therefore part of the skin sensor such that the response light is detected directly within the skin sensor, in contrast, for example, to the fiber-optic construction in U.S. Pat. No. 6,995,019 B2, in which an external detector is required.

In some aspects, the response light represents an optical response of the indicator substance to the incidence of the interrogation light. Accordingly, the detector and/or the detector in interaction with at least one response filter is configured to detect in a targeted manner in the spectral range of the response light. In some aspects, the detector and/or the detector in interaction with the at least one response filter is configured to suppress light outside the spectral range of the response light. In some aspects, the detector and/or the detector in interaction with the at least one response filter can be designed to suppress the interrogation light. In yet another aspect, response filters are designed to suppress the detection of ambient light, particularly at wavelengths that can travel long distances in tissue prior to absorption, such as a spectral range of from about 700 to about 1100 nm. The interrogation light and the response light can be configured such that they are spectrally different or spectrally shifted relative to one another with regard to their spectral intensity distribution.

By way of example and not limitation, in some aspects, the response light shifts toward longer wavelengths in comparison with the interrogation light, which generally occurs in a fluorescence measurement (i.e., the Stokes shift). By way of another example, the Stokes shift of a peak wavelength of the response light relative to a peak wavelength of the interrogation light is between about 10 nm and about 200 nm, more particularly between about 100 nm and about 150 nm, and particularly about 120 nm. The detector and/or the detector in interaction with the at least one response filter can be designed to detect such response light. About in this context means±10 nm.

The at least one radiation source, more particularly, the at least one light source, and the at least one detector are designed to irradiate the body surface with the interrogation light and to detect at least one response light incident from the direction of the body surface. The radiation source and the detector are therefore optically connected to the body surface in such a way that, through the body surface, for example transcutaneously, the interrogation light can be radiated into the body tissue or the body fluid of the patient, and that, likewise through the body surface, for example transcutaneously, the response light from the body tissue or the body fluid is observed by the detector.

In addition to the at least one detector and the at least one radiation source, the sensor assembly may comprise further elements. In some aspects, the attachment collar comprises further elements. In some aspects, the skin sensor comprises further elements. In some aspects, both the skin sensor and the attachment collar comprise further elements.

In some aspects, the sensor assembly further comprises a controller. The controller is programmed to control the at least one skin sensor comprising the at least one radiation source and the at least one detector.

Furthermore, the controller can be configured, for example, for driving or controlling the at least one radiation source and the at least one detector, for example, for starting an emission of the interrogation light and/or for initiating a detection of the response light. For this purpose, the controller can comprise, for example, corresponding drivers for the detector and/or the radiation source. A timing for a measurement can also be predefined, such that, for example, the controller can predefine a specific time scheme for the light source and/or the detector, said time scheme allowing a temporal sequence of the emission of the interrogation light and the detection of the response light. By way of example and not limitation, the controller can be designed to carry out or to control a temporally resolved measurement of the skin sensor. In this case, a measurement comprises the emissions of at least one interrogation light, more particularly of at least one pulse of the interrogation light, and the detection of at least one response light, more particularly of at least one pulse of the response light. A temporally resolved measurement can accordingly be understood to be a measurement in which, in addition, a time of the detection of the response light also plays a part or is registered. Thus, by way of example and not limitation, for each value of the response light, it is also possible to register the corresponding points in time at which this value is recorded and/or it is possible for the response light only to be recorded at specific points in time (gating). In this way, by means of temporally resolved measurements, for example, it is possible to obtain information about the rate in which an indicator substance is eliminated from the body of a patient via the kidneys. In some aspects, the detector is configured to detect the different time points generated by the interaction of an indicator substance with a light generated by the light source. In some aspects, the light source is modulated rather than pulsed, and the detected signal is selectively amplified or digitally demodulated to selectively detect signals at the frequency of the source. In some aspects, the connection between the controller and the skin sensor is cabled, wireless or a combination thereof. In some aspects, the connection between the controller and the other components is by a cable. In some aspects, the connection between the controller and the other components is wireless. In some aspects, the controller is contained within the sensor assembly.

Furthermore, the sensor assembly may further comprise a processor. The processor may be designed to carry out partial or complete processing of the measurement results. In particular, in this case it is possible to process the signals recorded by the at least one detector, and optionally additional information such as, for example, time information, for example the points in time at which the measurement signals of the detector were recorded. The measurement values or measurement signals of the detector can be, for example, intensities of the response light and/or signals of electrical type which correlate with said intensities. In this case, by way of example, complete or partial processing of these signals can be effected, such that, for example, filtering, smoothing, averaging or the like is already effected in the processor. Alternatively or additionally, an evaluation of these signals can also already be effected at least in part, for example a determination of a waveform and/or of a half-life and/or a determination of an indicator substance concentration corresponding to these signals. In some aspects, the connection between the processor and the skin sensor is cabled, wireless or a combination thereof. In some aspects, the connection between the processor and the other components is by a cable. In some aspects, the connection between the processor and the other components is wireless. In some aspects, the processor is contained within the sensor assembly. In some aspects, the controller and the processor are the same device. In some aspects, the processor is an integrated component in the controller.

Partial or complete storage of the information in the sensor assembly, more particularly in the processor, is also conceivable. Said information can comprise, for example, one or a plurality of detector signals or information derived therefrom, time information, information about the interrogation light, for example an intensity of the interrogation light, or combinations of said information and/or further information. In order to store the information, the sensor assembly, more particularly the processor, can comprise for example one or a plurality of data storage devices, more particularly volatile and/or nonvolatile data memories. Generally, the processor can be configured wholly or partly using electrical components, wherein one or a plurality of data processing units, for example microprocessors and/or ASICs, can also be used.

In some aspects, the sensor assembly comprises an authentication system programmed to receive authentication information from the skin sensor, the attachment collar or both. The authentication technology comprises techniques known in the art such as, for example, EEPROM or RFID (radiofrequency identification label) technology.

In some aspects, the skin sensor may further comprise, for example, at least one interface for data exchange. Said data can be, for example, measurement results for intensities of the response light detected by the detector. Data already partly processed, filtered or partly or completely evaluated data, can also be transmitted via said interface. The interface can be configured as a wireless interface, a cabled interface or a combination thereof, and can comprise a radiofrequency coil and/or a cable. In some aspects, transponder technology known in the art may be used, for example, to initiate a measurement via the skin sensor and/or to interrogate measurement data from the skin sensor. In some aspects, corresponding radiofrequency readers such as are known from RFID technology, for example, can be used for this purpose.

In some aspects, the sensor assembly further comprises a cable management system configured to reduce or eliminate accidental cord pulls that would dislodge or detach the sensor assembly from the body of the patient and/or reduce or eliminate accidental cord pulls that would dislodge or detach the skin sensor from the attachment collar, said cable management system is attached to the attachment collar, the skin sensor or both.

In order to reduce possible light transmission through the skin sensor and the attachment collar, in some aspects, one or both are fabricated out of elastomeric materials. In some aspects, the elastomer is mixed with graphite and/or carbon black and/or other light-absorbing materials. In some aspects, an optically non-transmissive material is included as a layer. In some aspects, the optically non-transmissive material is mylar. Mylar is highly absorptive of UV, visible and near infrared light while also being thin and flexible. In another aspect, the optically non-transmissive material is aluminum. Aluminum is also highly absorptive of UV, visible and near infrared light while also being thin and flexible. This reduces light transmission through the skin sensor and/or attachment collar. In some aspects, both the skin sensor and the attachment collar are fabricated from an elastomer that is mixed with graphite, carbon black or a combination thereof. An optically non-transmissive material is one that reduces or eliminates the passage of light therethrough. In some aspects, the passage of light is entirely eliminated. In some aspects, the passage of light is reduced by about 99%, by about 98%, by about 97%, by about 96%, by about 95%, or by about 90%. About as used in this context means±1%. In some aspects, the attachment collar is disposable.

In some aspects, the skin sensor and attachment collar are designed to ameliorate the effects of accumulation of excess fluid within the skin of the patient beneath the sensor, which could otherwise have detrimental or undesirable effects on the sensor measurements. In some aspects, where the rate of elimination of an exogenous agent is being measured, variation over time in the fractional volume of interstitial fluid within the measured tissue volume may result in uncertainty and/or inaccuracy in the transdermally measured elimination rate. Such may be the case when the sensor is placed over an area that is locally edematous, or in patients with whole-body excess fluid build-up (“fluid over-load”) such as is common in patients with, for example, compromised kidney function or congestive heart failure. Such excess fluid may be removed from the field of measurement by the application of light pressure against the skin (e.g., 10-20 mm Hg), without exsanguinating the skin or shifting the balance of more tightly bound interstitial fluid. In some aspects, a positive pressure is exerted on the surface of the skin directly beneath the skin sensor, while simultaneously applying a negative pressure on the surrounding skin surface, beneath which the attachment collar is mounted. In some aspects, this is accomplished by first securely mounting the attachment collar to the skin, then mounting the skin sensor into the collar such that the sensor protrudes slightly beyond the collar, thereby pressing more firmly against the skin beneath the sensor, with a compensating negative pressure in the area beneath the attachment collar.

In some aspects, a 2-sided adhesive is employed within an aperture inside the attachment collar. The side facing the skin is selected to adhere reliably to the skin for an extended period of time (e.g., 24 to 48 hrs.), even in the presence of moisture, such as sweat. In some aspects, an acrylate-based adhesive is used for bonding to the skin. In yet another aspect, the skin is pre-treated with a barrier film, such as by application of rapidly-drying liquid film that upon drying forms a “second skin”. In such aspects the barrier film aids in the long-term, reliable attachment of the acrylate-based adhesive to the skin, while also having the benefit of allowing sensor removal without disruption or removal of the skin epidermis. In some aspects, the barrier film is CAVILON™ (manufactured by 3M). The second side of the adhesive, which faces towards the sensor, may be selected to adhere as strongly as desired to the face of the sensor. In one such aspect the sensor face is constructed from a polymer material, such as MAKROLON™, and the adhesive is rubber based. One non-limiting example of an appropriate 2-sided adhesive is 3M product #2477 (Double-Coated TPE Silicone Acrylate Medical Tape with Premium Liner).

In some aspects, the adhesive bond formed between the attachment collar and sensor is relatively weak or even non-existent until the adhesive is placed under mild pressure. Such embodiments have the additional benefit of forming a secure interface between the sensor and the skin when the sensor is placed under positive pressure against the skin, but once released, the sensor is easily removed from the attachment collar without leaving a residue on the sensor. In some aspects where the sensor is reusable and the collar is disposable or single use, the sensor portion never contacts the skin. If the sensor does not come into direct contact with the skin of the patient, this reduces the chance of contamination and reduces the cleaning and/or sterilization needed before the sensor is reused on the same or a different patient.

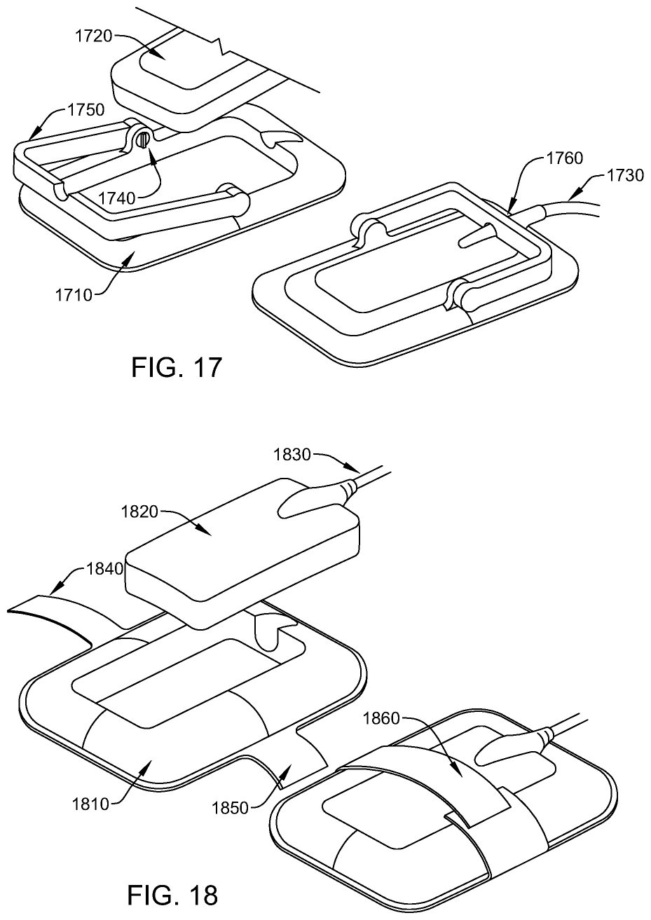

The above-described advantages for the sensor that applies a small positive pressure over the skin area under measurement may be combined with the also above-described aspect wherein a small positive pressure is required to adhere the sensor to the attachment collar. A nonlimiting example that illustrates these advantages is shown in in FIG. 17. These same advantages and features may also be incorporated into other embodiments illustrated herein.

In some aspects, it is desirable that a method for reliably or securely identifying or authenticating the attachment collar is provided. In some aspects the collar includes an encrypted identifier or identification tag that prevents the use on non-approved devices. In some aspects the encryption code is embedded in an EEPROM chip within the attachment collar. Use of the sensor is prevented unless a connection is made between the sensor and collar, and the collar is identified as being valid. In other aspects, the EEPROM is used to identify a particular product version, mode of operation, and/or algorithm coefficients for instrument operation. In this manner, different functions of the sensor may be enabled through the EEPROM coding.

In some aspects, the attachment collar further comprises a pressure sensitive element that communicates with the sensor when attached. In some aspects, the pressure sensor provides an indication of secure attachment of the skin sensor to the skin of the patient. In some aspects, the indication that the sensor is no longer securely attached is used to discontinue measurement, and/or to provide feedback to a user.

In some aspects, the attachment collar is intended for single use and the pressure sensor is used to enforce this. In some aspects, the pressure sensor determines that the attachment collar has been placed on a patient, and then determines that it has been subsequently removed. Any subsequent attempts to reuse the sensor with the same attachment collar are prevented.

In some aspects, the sensor assembly may further comprise a removable cover at least partially enclosing the skin sensor. The cover may be configured to protect the skin sensor from external elements, such as moisture, debris and the like. The cover may be manufactured from the same material as the attachment collar or any other material suitable to provide adequate protection for the skin sensor.

In some aspects, the sensor assembly may comprise a skin sensor disposed in an opening of a sensor housing that may include an attachment collar and a cover. In some aspects, the sensor housing may include an attachment collar removably attached to the cover. In other aspects, the sensor housing may include an attachment collar integrally attached to the cover to form one piece. The sensor housing may be configured to allow for independent movement of the skin sensor relative to the sensor housing. In some aspects, the skin sensor may be configured to move, slide, pivot, tilt or rotate independently of the sensor housing in response to external forces placed on the sensor assembly in any direction. In some aspects, the skin sensor may be configured to slide independently in an upward and downward direction relative to the sensor housing in response to negative and positive pressures placed on the sensor assembly, respectively. In some aspects, the skin sensor may be configured to rotate independently relative to the sensor housing in response to a rotational force placed on the sensor assembly.

In some aspects, the sensor assembly may be configured to stay in contact with the surface of the skin of a patient and remain in contact with the skin even when external forces are applied to the sensor assembly. In some aspects, the skin sensor of the sensor assembly may be configured to move, slide, pivot, tilt or rotate internal to the sensor housing and independently of the sensor housing. In some aspects, the sensor assembly may include biasing assemblies that may assist in preventing the transfer of force to the skin sensor when an external force is applied to the sensor assembly. Suitable biasing assemblies include spring assemblies, damper members, foam materials, rubber or other elastomeric materials, or any other assembly that assists in preventing the transfer of force to the skin sensor when an external force is applied to the sensor assembly.

In some aspects, the skin sensor may be configured to move, slide, pivot, tilt or rotate without the aid of biasing assemblies. In some aspects, the skin sensor may be configured to rest on the surface of the skin of the patient and float inside the sensor housing in response to changes in either internal and/or external force.

In some aspects, the attachment collar and skin sensor may further comprise an adhesive layered on at least a portion of a bottom surface of one or each of the attachment collar and skin sensor. The adhesive may be configured to more securely attach the sensor assembly to the skin of a patient. Suitable adhesives may include materials configured to reliably adhere to the skin for an extended period of time (e.g., 24 to 48 hrs.), even in the presence of moisture, such as sweat. In some aspects, an acrylate-based adhesive is used for bonding to the skin.

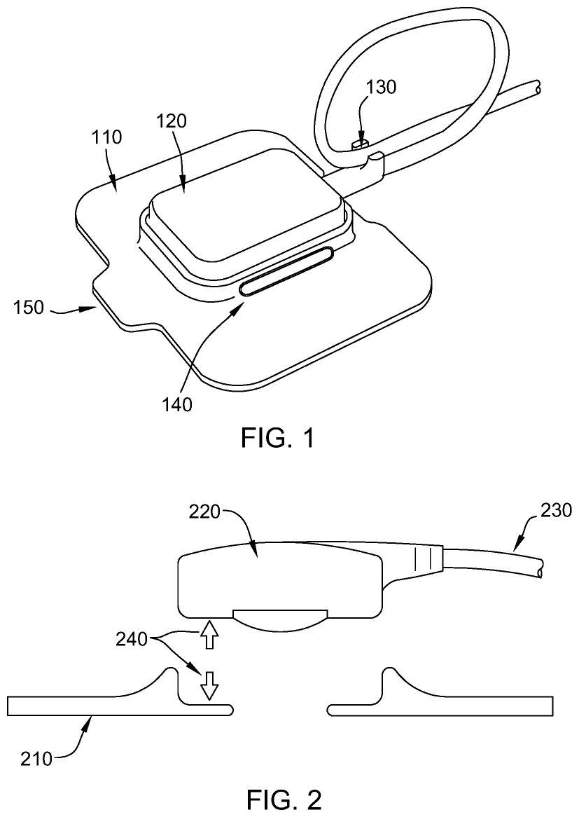

With reference to FIG. 1, the sensor comprises an attachments collar 110 and a sensor 120. The cable is clipped into a cable management system 130 to reduce cord pulls and provide strain relief. Sidebars 140 secure sensor 120 to attachment collar 110, while pull tab 150 allows for easy removal of attachment collar 110 from the skin of the patient after use.

With reference to FIG. 2, the sensor comprises an attachment collar 210 and a sensor 220. Cable 230 is coupled to a controller that can send and receive information therebetween. Also shown and represented by the arrows is a selective adhesive 240 that secures sensor 220 to attachment collar 210.

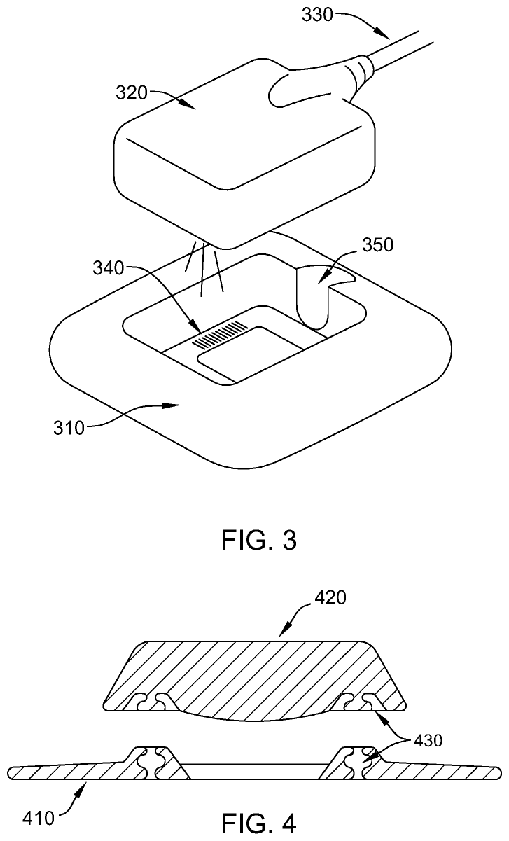

With reference to FIG. 3, the sensor comprises an attachment collar 310 and a sensor 320. Cable 330 is coupled to a controller that can send and receive information therebetween. Also shown is a bar code 340 that is used to authenticate the combination of sensor 320 and attachment collar 310 thereby ensuring that a light-tight fit and secure attachment to the patient's body surface is achieved. Also shown is cable management groove 350 to help reduce the opportunity for the cable to become caught and dislodged from the patient.

With reference to FIG. 4, the sensor comprises an attachment collar 410 and a sensor 420. Also shown is one possible aspect of a light-tight connector 430 between sensor 420 and attachment collar 410. The non-linear surfaces of light-tight connector 430 reduces and/or eliminates extraneous light that may leak through between the interface of sensor 420 and attachment collar 410.

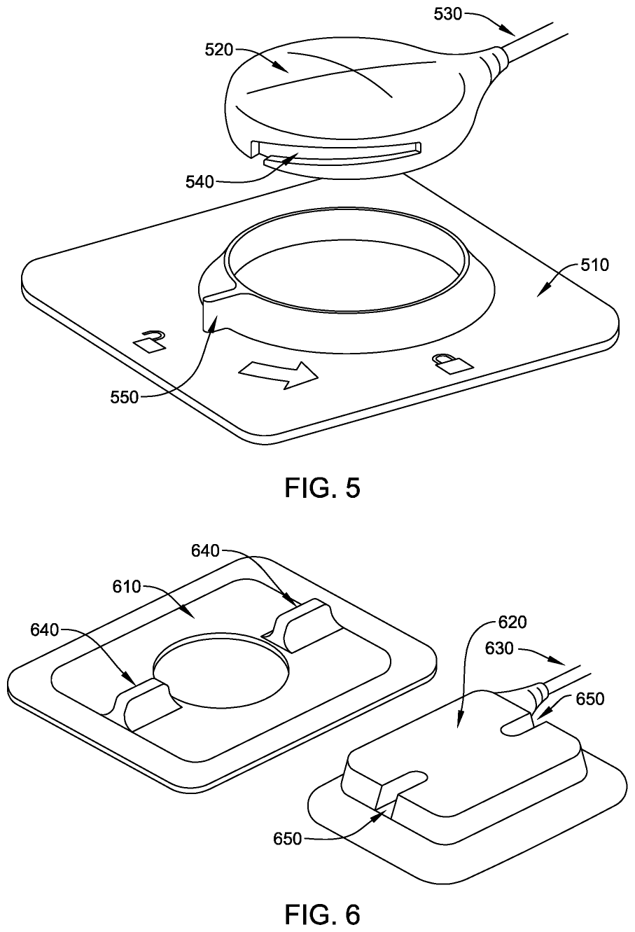

With reference to FIG. 5, the sensor comprises an attachment collar 510 and a sensor 520. Also shown is one possible cam locking mechanism 530 that would secure sensor 520 to attachment collar 510. Sensor 520 would slidably connect tab 550 to slot 540 and then twist to secure the sensor in place ensuring that a secure attachment to the patient's body surface is achieved. This locking mechanism would also be light-tight due to the nonlinear aspect of the male and female ends of the lock.

With reference to FIG. 6, the sensor comprises an attachment collar 610 and a sensor 620. Cable 630 is coupled to a controller that can send and receive information therebetween. In this aspect, tabs 640 fit into slots 650 thereby ensuring proper alignment of sensor 620 with attachment collar 610 ensuring that a secure attachment and light-tight fit to the patient's body is achieved.

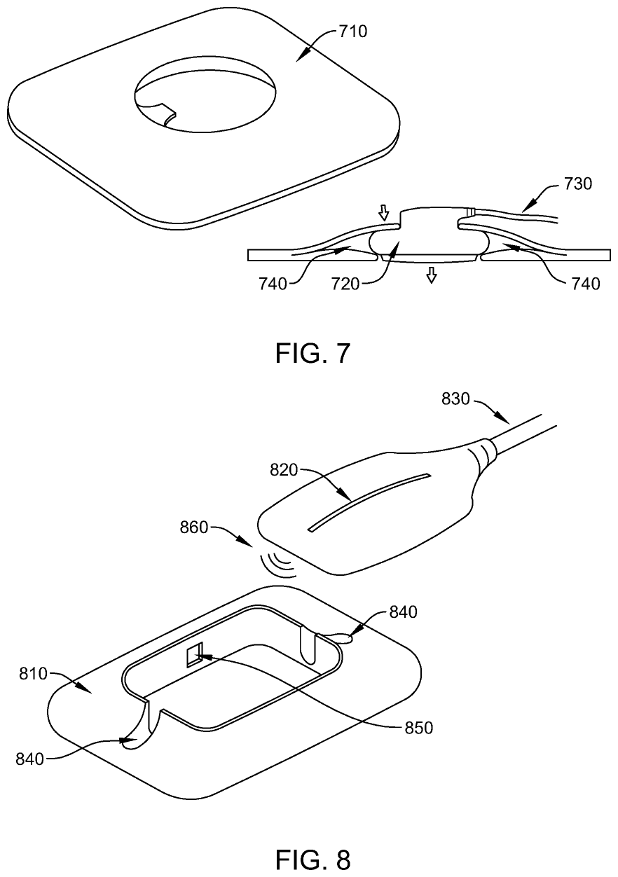

With reference to FIG. 7, the sensor comprises an attachment collar 710 and a sensor 720. Cable 730 is coupled to a controller that can send and receive information therebetween. In this aspect, attachment collar 710 is a stretchable pocket that includes an internal cavity 740 therein that receives sensor 720 ensuring a light-tight fit.

With reference to FIG. 8, the sensor comprises an attachment collar 810 and a sensor 820. Cable 830 is coupled to a controller that can send and receive information therebetween. In this aspect, slot 840 could receive and secure cable 830 thereby reducing the incidence of cord-pull by the patient. Also shown is an RFID chip 850 that includes a security code that must be detected 860 by the sensor in order for the system to operate. This RFID code can be used for device security, for ensuring that a secure attachment and light-tight fit to the patient's body is achieved, and for inventory control.

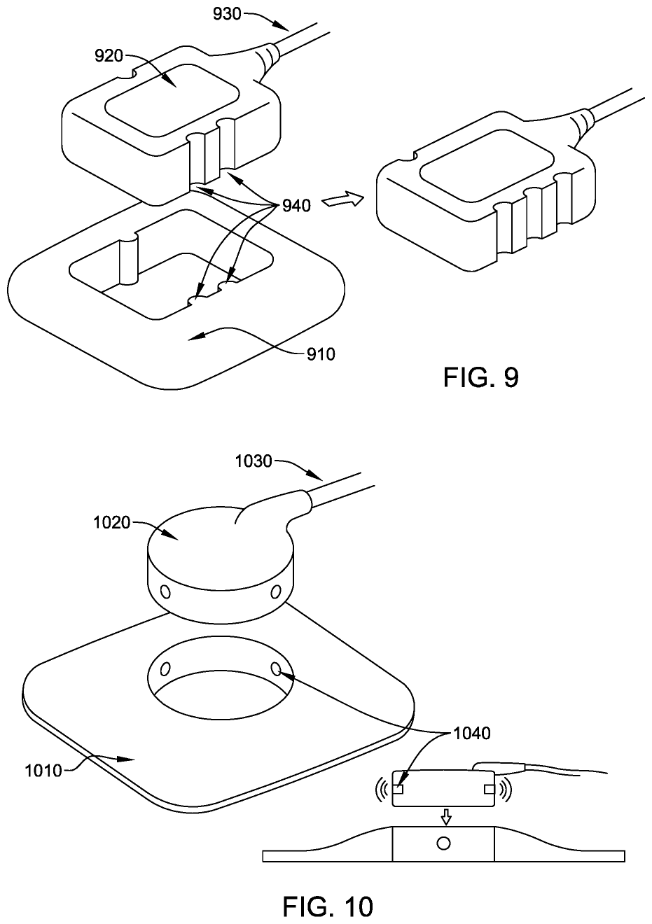

With reference to FIG. 9, the sensor comprises an attachment collar 910 and a sensor 920. Cable 930 is coupled to a controller that can send and receive information therebetween. In this aspect, slots 940 are present to ensure a proper connection between sensor 920 and attachment collar 910. The shape of the slots can be varied in the manner of a lock and key to ensure a secure attachment and as a form of fraud prevention and quality control.

With reference to FIG. 10, the sensor comprises an attachment collar 1010 and a sensor 1020. Cable 1030 is coupled to a controller that can send and receive information therebetween. In this aspect, magnets 1040 are present to secure sensor 1020 to attachment collar 1010.

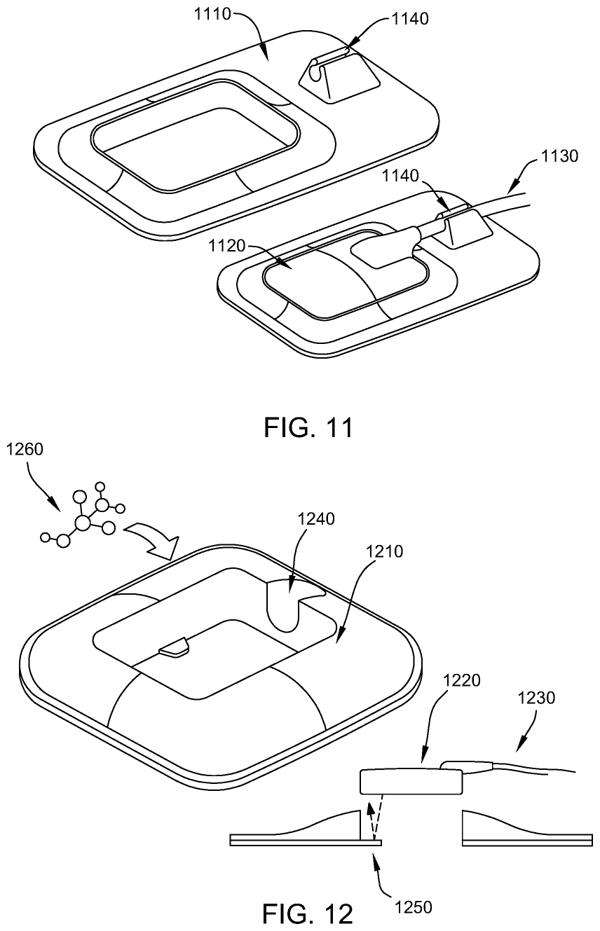

With reference to FIG. 11, the sensor comprises an attachment collar 1110 and a sensor 1120. Cable 1130 is coupled to a controller that can send and receive information therebetween. In this aspect, cable clip 1140 is on attachment collar 1110 to secure cable 1130 to reduce or eliminate the incidence of cord-pull that would interfere with the use of the system.

With reference to FIG. 12, the sensor comprises an attachment collar 1210 and a sensor 1220. Attachment collar 1210 comprises an embedded chemical 1260 that is detected by sensor 1220 when properly placed to provide a secure connection. Cord 1230 seats into slot 1240 thereby reducing movement and play in the cord. Also included is identifier tab 1250 that is used to authenticate the attachment collar 1210 after detection by sensor 1220.

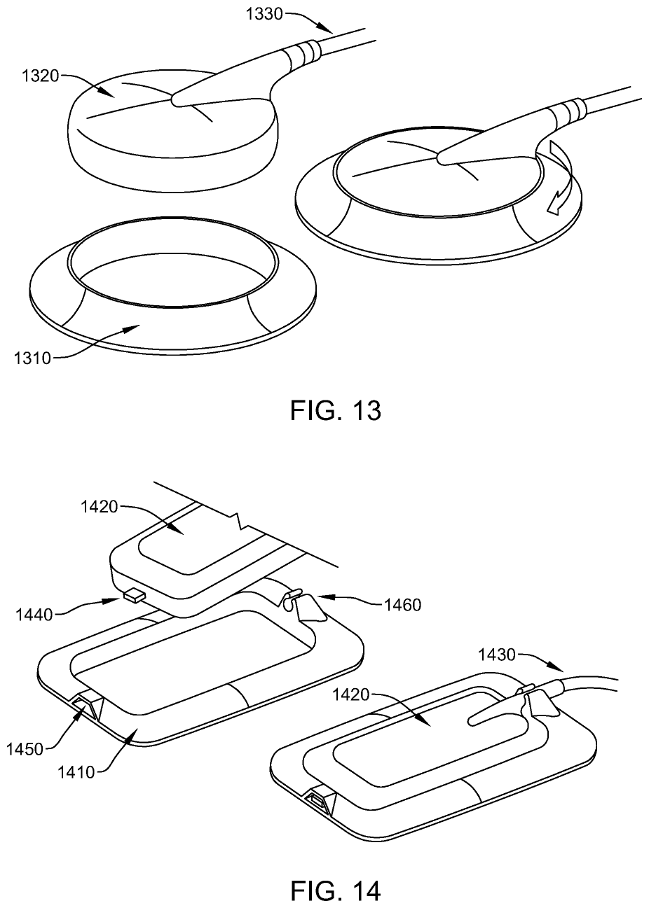

With reference to FIG. 13, the sensor comprises an attachment collar 1310 and a sensor 1320. Cable 1330 is coupled to a controller that can send and receive information therebetween. In this aspect, sensor 1320 screws into attachment collar 1310 to provide a secure connection and light-tight interface between them.

With reference to FIG. 14, the sensor comprises an attachment collar 1410 and a sensor 1420. Cable 1430 is coupled to a controller that can send and receive information therebetween. In this aspect, tab 1440 fits into slot 1450 to secure sensor 1420 to attachment collar 1410. Cable 1430 fits into cable clip 1460 to secure the cable and reduce or eliminate the incidence of cord-pull that would interfere with the use of the system.

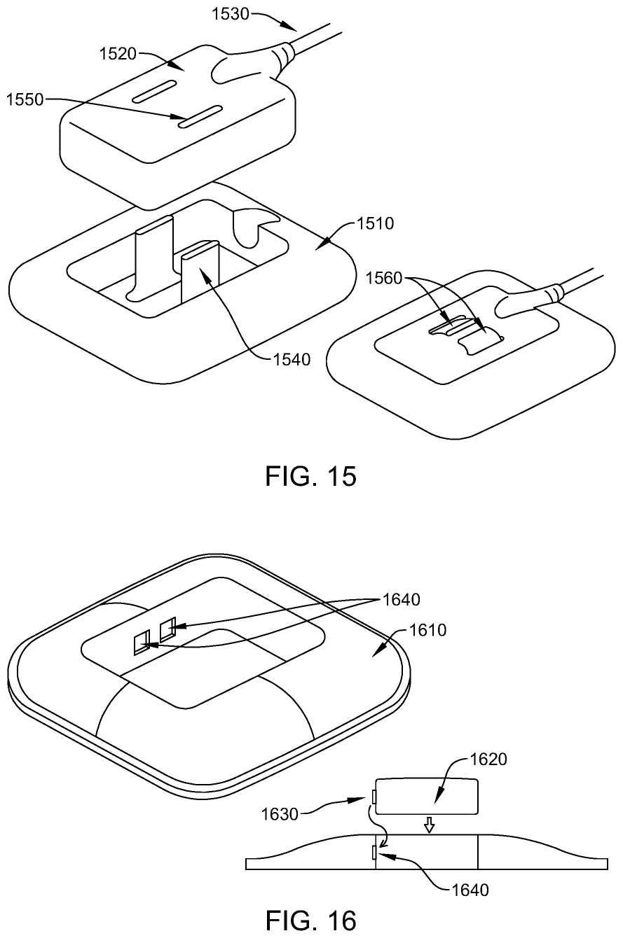

With reference to FIG. 15, the sensor comprises an attachment collar 1510 and a sensor 1520. Cable 1530 is coupled to a controller that can send and receive information therebetween. In this aspect, tabs 1540 fit through slots 1550 on sensor 1520 and fold over 1560 to secure sensor 1520 to attachment collar 1510 ensuring that a secure attachment and light-tight fit to the patient's body is achieved.

With reference to FIG. 16, the sensor comprises an attachment collar 1610 and a sensor 1620. The cable (not shown) is coupled to a controller that can send and receive information therebetween. In this aspect, tabs 1630 would insert into holes 1640 thereby ensuring a secure connection between sensor 1620 and attachment collar 1610. Holes 1640 could also be communication ports to send and receive information between sensor 1620 and attachment collar 1610 for both security and inventory purposes. This would eliminate the need for a wireless authentication thereby reducing the complexity and electronic components required in the overall system. In some aspects, communication between sensor 1620 and attachment collar 1610 is via an EPROM type memory ensuring that a secure attachment and light-tight fit to the patient's body is achieved.

With reference to FIG. 17, the sensor comprises an attachment collar 1710 and a sensor 1720. Cable 1730 is coupled to a controller that can send and receive information therebetween. In this aspect, a cam-lock 1740 secures sensor 1720 to attachment collar 1710 after engaging locking arm 1750. Simultaneously, when locking arm 1750 is engaged to secure sensor 1720, it also secures cable 1730 in a secure configuration 1760 ensuring that a secure attachment and light-tight fit to the patient's body is achieved.

With reference to FIG. 18, the sensor comprises an attachment collar 1810 and a sensor 1820. Cable 1830 is coupled to a controller that can send and receive information therebetween. Sensor 1820 is securely attached to attachment color 1810 via a strap 1840 that engages with tab 1850. Engagement 1860, in some aspects, can be using Velcro, an adhesive, snap, buckle or other appropriate means ensuring that a secure attachment and light-tight fit to the patient's body is achieved.

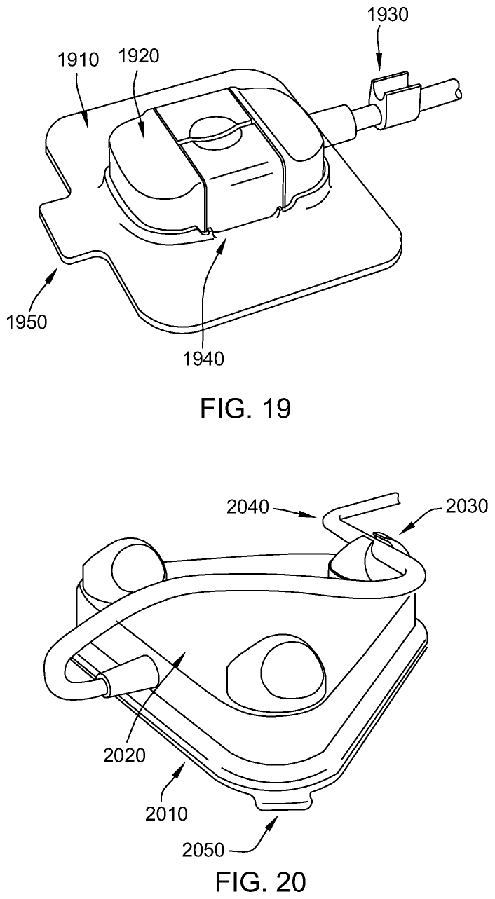

With reference to FIG. 19, the sensor comprises an attachment collar 1910 and a sensor 1920. The cable includes a cable management clip 1930 to secure the cord and reduce cord pulls and provide strain relief. Side clips 1940 secure sensor 1920 to attachment collar 1910 while pull tab 1950 allows for easy removal from the skin of a patient after use.

With reference to FIG. 20, the triangular shaped sensor comprises an attachment collar 2010 and a sensor 2020. Cable 2040 clips into cable management system 2030 to reduce cord pulls and provide strain relief. Pull table 2050 allows for easy removal from the skin of a patient after use.

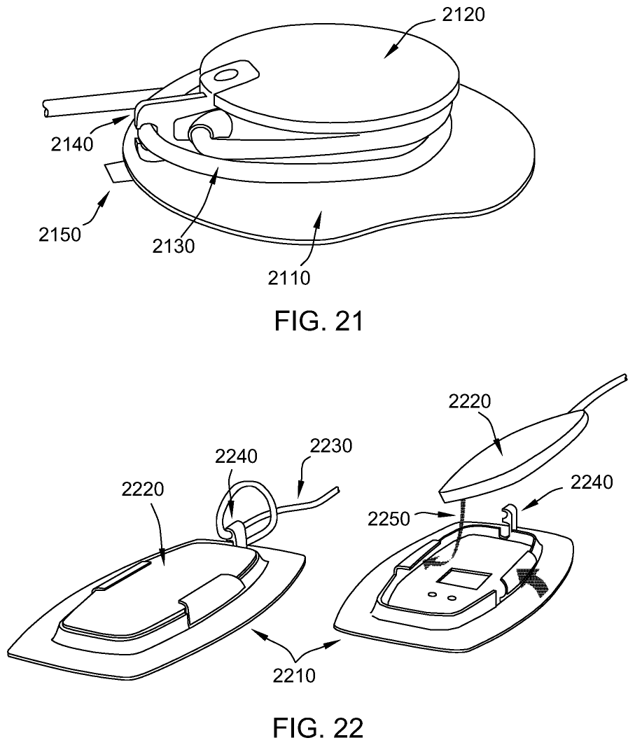

With reference to FIG. 21, the sensor comprises an attachment collar 2110 and a sensor 2120. Cable 2130 wraps around skin sensor 2020 and clips into cable management system 2140 to reduce cord pulls and provide strain relief. Pull table 2150 allows for easy removal from the skin of a patient after use.

With reference to FIG. 22, the sensor comprises an attachment collar 2210 and a sensor 2220. Sensor 2220 slides under side table 2250 on the attachment collar 2210 to secure the sensor in place. Cable 2230 clips into cable management system 2240 to reduce cord pulls and provide strain relief. Pull table 2150 allows for easy removal from the skin of a patient after use.

In some aspects the attachment collar further comprises a means for securing the skin sensor to the at least one opening of the attachment collar as illustrated in FIGS. 1 to 22. In some aspects, the sensor assembly further comprises a means for managing a cable attached thereto as illustrated in FIGS. 1 to 22. In some aspects, the means for managing the cable is attached to the attachment collar, the skin sensor or both as illustrated in FIGS. 1 to 22. In some aspects, the skin sensor and/or the attachment collar further comprises a means of authentication between the skin sensor and the attachment collar as described elsewhere herein.

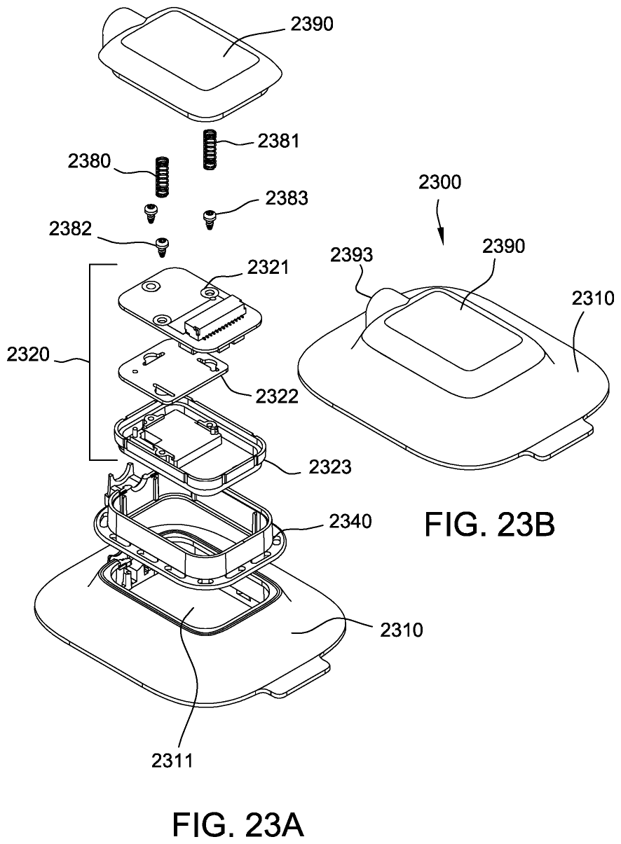

With reference to FIGS. 23A and 23B, the sensor assembly may include a movable skin sensor 2320 housed in a sensor housing 2300 including a cover 2390, and an attachment collar 2310. The cover 2390 may be removably attached to the attachment collar 2310, as shown in FIGS. 23A and 23B, or may be integrally connected to the attachment collar 2310 to form one piece. The movable skin sensor 2320 includes a skin sensor head 2321 secured to a sliding chassis 2323, with an optical gasket 2322 disposed therebetween. The sliding chassis 2323 may be slidably disposed in a frame 2340, which may be removably or integrally secured in an opening 2311 of attachment collar 2310. In some aspects, the sliding chassis 2323 may be slidably disposed directly in the opening 2311 of the attachment collar 2310 without the use of a frame 2340. In some aspects, the cover 2390 may be removably attached to the frame 2340. In other aspects, the cover 2390 may be integrally attached to the frame 2340 to form one piece.

The sliding chassis 2323 may be configured to allow for slidable movement of the movable skin sensor 2320 in and out of the attachment collar 2310 and assists in preventing the transfer of force to the skin sensor 2320 when an external force is applied to the cover 2390 and/or the attachment collar 2310. The sensor assembly of FIGS. 23A and 23B additionally includes a first spring 2380 and a second spring 2381 secured to the skin sensor 2320 using a first screw 2382 and a second screw 2383, respectively. The first spring 2380 and second spring 2381 may be positioned between the skin sensor 2320 and the cover 2390 and configured to prevent the transfer of force to the movable skin sensor 2320 when an external force is applied to the cover 2390. In various aspects, the external force may be applied in any vector, including but not limited to the side or top of the sensor assembly. In various aspects, any number of springs and any configuration or placement of the springs may be employed to prevent the transfer of force to the skin sensor 2320 when an external force is applied to the cover 2390 and/or the attachment collar 2310.

In various aspects, as shown in FIGS. 23A and 23B, the sensor assembly may further contain an attachment point 2393 for a cable (not shown) that is attached to the movable skin sensor 2320 and extends from the cover 2390 and attachment collar 2310. In various aspects, the movable skin sensor 2320 may be configured to move independently of the attachment collar 2310 and cover 2390 when an external force is applied to the cable, for example, when the cord is pulled. In other aspects, the sensor assembly may be configured as a wireless sensor assembly free of any cables or cords.

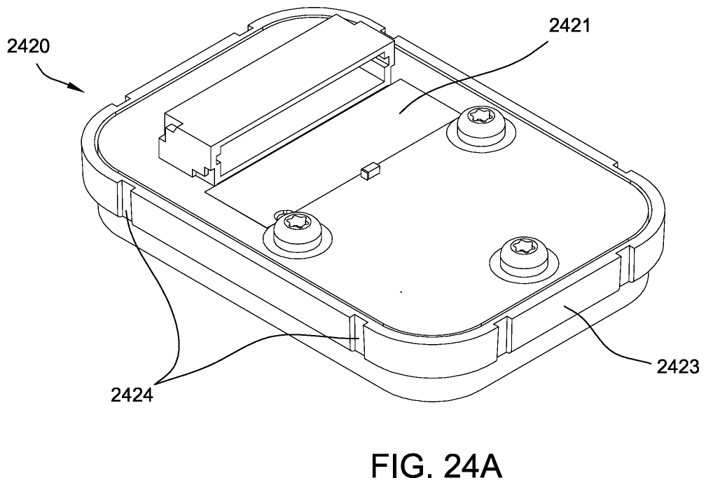

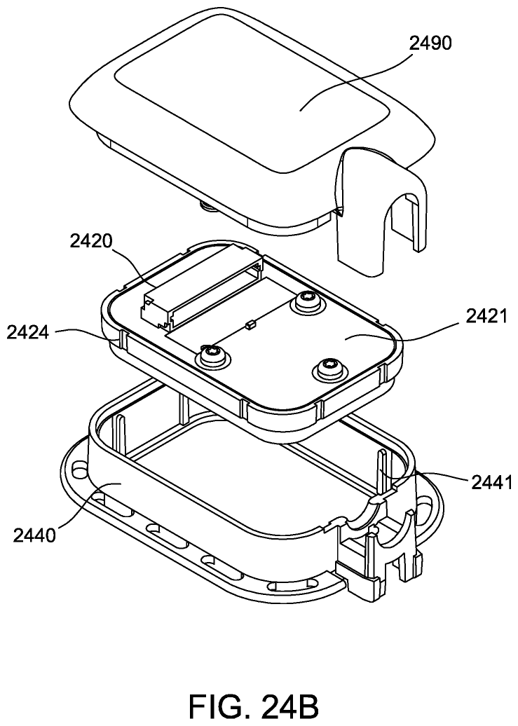

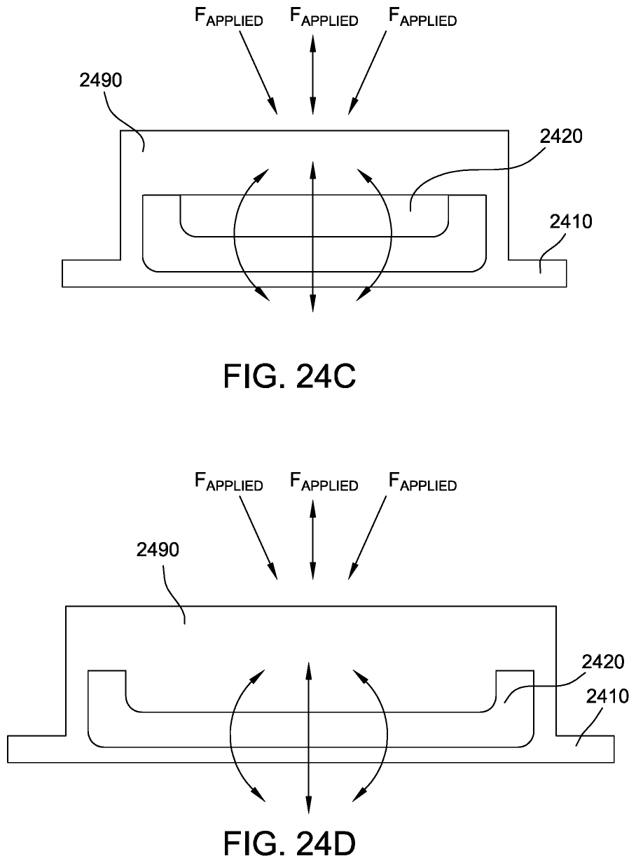

With reference to FIG. 24A, the sensor assembly includes a movable skin sensor 2420, which includes the skin sensor head 2421 disposed in the sliding chassis 2423. The sliding chassis 2423 includes a plurality of slots 2424 that are arranged around an outer perimeter of the sliding chassis 2423. With reference to FIG. 24B, the sensor assembly may include frame 2440, which may be configured to slidably receive movable skin sensor 2420. In various aspects, the frame 2440 may be configured to be removably or integrally secured in an opening of attachment collar 2310, as shown in FIG. 23A.

As shown in FIG. 24B, the cover 2490 is configured to fit over the movable skin sensor 2420. The cover 2490 may be further configured to be removably or integrally connected to the frame 2440 and/or attachment collar 2310 (FIG. 23A). The frame 2440 may include a plurality of ribs 2441 arranged around an inner perimeter of the frame 2440. The slots 2424 located on the outer perimeter of the sliding chassis 2423 are configured to slidably mate with congruent ribs 2441 located on the inner perimeter of the frame 2440. The mated slots 2424 and ribs 2441 are configured to allow for a sliding movement of the movable skin sensor 2420 in an upward and downward direction relative to the frame 2440 in response to negative and positive pressures placed on the sensor assembly, respectively. In some aspects, the slots 2424 are configured to allow for additional degrees of freedom of movement of the ribs 2441 within the slots 2424, for example, by configuring the slots 2424 to be larger in width than the ribs 2441. In some aspects, one or more of the slots 2424 may be configured to allow for tilting of the skin sensor 2420 in at least one direction. In some aspects, all of the slots 2424 may be configured to allow for tilting of the skin sensor 2420 in all directions. Exemplary additional degrees of freedom of movement imparted by the slots 2424 and ribs 2441 are illustrated in FIGS. 24C and 24D.

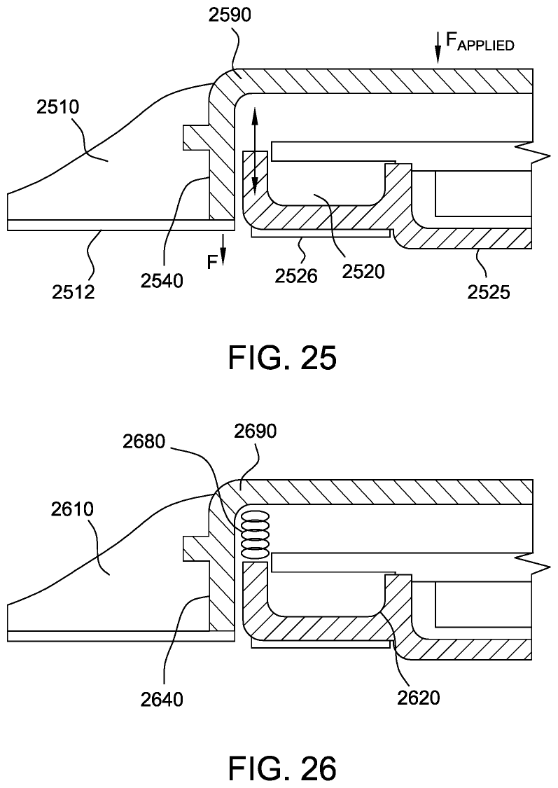

With reference to FIG. 25, the sensor assembly includes a movable skin sensor 2520 configured to move in an upward and downward direction independently of frame 2540 disposed in an opening of attachment collar 2510, and independently of cover 2590. In various aspects, the cover 2590 may be integrally attached to the frame 2540 and/or the attachment collar 2510, as shown in FIG. 25. In various aspects, the cover 2590 may be removably attached to the frame 2540 and/or the attachment collar 2510. The movable skin sensor 2520 is configured to maintain its position on the skin of a patient even when an external downward force is exerted on the attachment collar 2510 and/or cover 2590. In some aspects, the movable skin sensor 2520 may be mounted in the attachment collar 2510 such that the movable skin sensor 2520 protrudes slightly beyond the collar to form protrusion 2525, thereby pressing more firmly against the skin beneath the sensor, with a compensating negative pressure in the area beneath the attachment collar 2510. The movable skin sensor 2520 may include a first adhesive layer 2526 on a portion of the bottom surface of the movable skin sensor 2520 surrounding the protrusion 2525. The first adhesive layer 2526 may be configured to fix the movable skin sensor 2520 more securely in place on the skin of the patient and prevent movement of the movable skin sensor 2520 relative to the cover and/or attachment collar. By way of example, the first adhesive layer 2526 may be configured to fix the movable skin sensor 2520 in place when negative pressure is applied to the sensor assembly (i.e. cord pull), wherein the movable skin sensor 2520 remains secured to the skin of the patient while permitting movement of the attachment collar 2510 and/or cover 2590 when the cord is pulled. Further by way of example, the first adhesive layer 2526 may allow for the movable skin sensor 2520 to slide downward and extend beyond attachment collar 2510 to keep the movable skin sensor 2520 in communication and/or contact with the skin of the patient even when a positive or negative force is exerted on the attachment collar 2510 and/or cover 2590. The attachment collar 2510 may further include a second adhesive layer 2512 configured to more securely fix the sensor assembly to the skin of the patient.

With reference to FIG. 26, the sensor assembly includes a movable skin sensor 2620 configured to move independently of frame 2640, wherein the sensor assembly includes a spring 2680 positioned between the movable skin sensor 2620 and the cover 2690. The spring 2680 is configured to further prevent the transfer of a positive or negative force exerted on the cover 2690 to the movable skin sensor 2620. In various aspects, the spring 2680 may be configured to exert any level of spring pressure sufficient to prevent the transfer of force exerted on the cover 2690 to the movable skin sensor 2620. In some aspects, the spring 2680 may be configured to exert a light spring pressure on a top surface of the skin sensor 2620. In some aspects, the spring 2680 may be characterized by a low k constant.

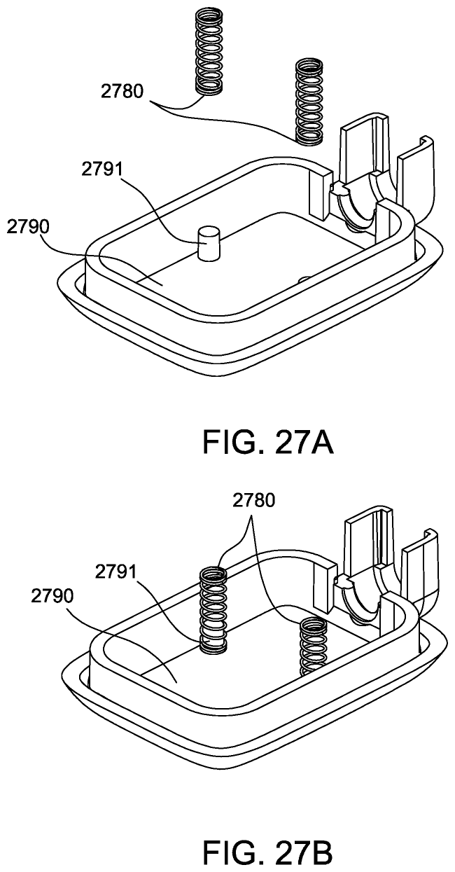

With reference to FIGS. 27A-B the sensor assembly includes cover 2790 which may include posts 2791 configured to accommodate springs 2780 and further configured to secure springs 2780 in a fixed position between the cover 2790 and the movable skin sensor 2620. The posts 2791 may be cylindrical in shape to accommodate springs 2780. However, in various aspects, the posts 2791 may be configured in any shape or configuration suitable to secure a biasing assembly to the cover 2790.

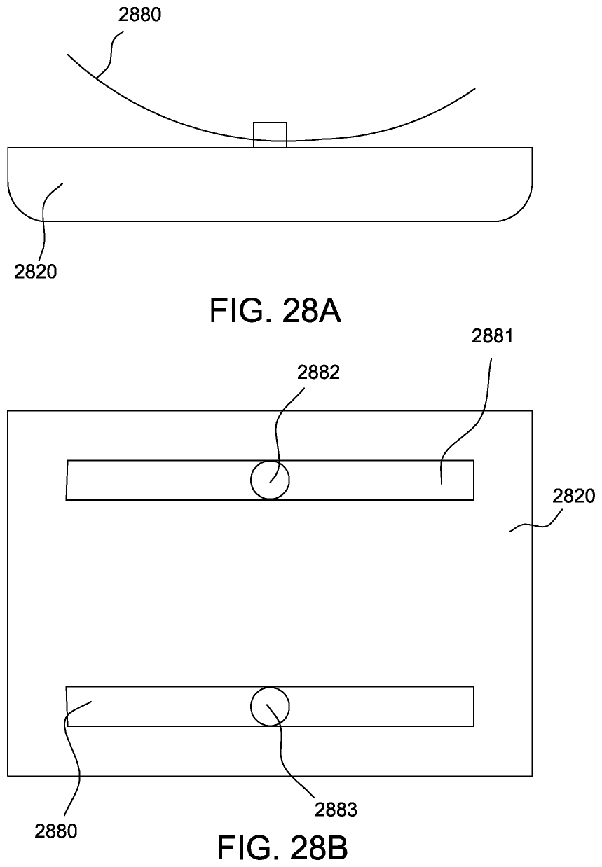

With reference to FIGS. 28A and 28B, the sensor assembly includes an alternative spring assembly that includes leaf springs 2880 and 2881. Leaf springs 2880 and 2881 may be positioned opposite each other and secured using screws 2882 and 2883 positioned on a top surface of the movable skin sensor 2820. In some aspects, leaf springs 2880 and 2881 may also be positioned on a bottom surface of the cover 2790 (FIG. 27B), and, for example, secured to posts 2791. Leaf springs 2880 and 2881 may be manufactured from any suitable material, including plastic materials, metallic materials, and the like.

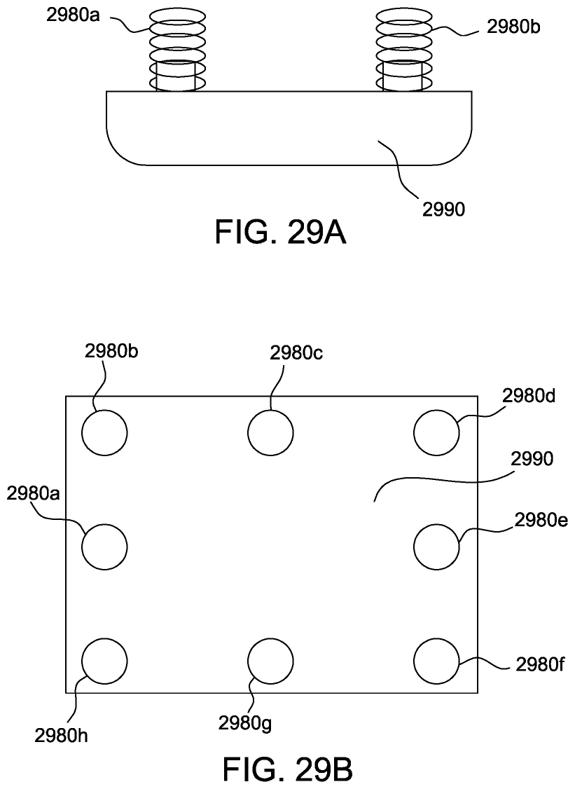

With reference to FIGS. 29A and 29B, the sensor assembly includes an alternative spring assembly, wherein additional springs 2980a-2980h are positioned around a perimeter of the cover 2990. In various aspects, the additional springs 2980a-2980h may be secured to the cover 2990 using posts 2791 as shown in FIG. 27A. In various aspects, the additional springs 2980a-2980h may alternatively be secured around a perimeter of the movable skin sensor 2320 (FIG. 23A) and further secured to corresponding posts (not shown) on cover 2990. In various aspects, the movable skin sensor 2320 may also include post-like projections configured to secure the additional springs 2980a-2980h to the movable skin sensor 2320. In other aspects, any number and positioning of additional springs may be used. The spring assembly of FIGS. 29A and 29B may allow for additional prevention of transfer of force to the movable skin sensor 2320.

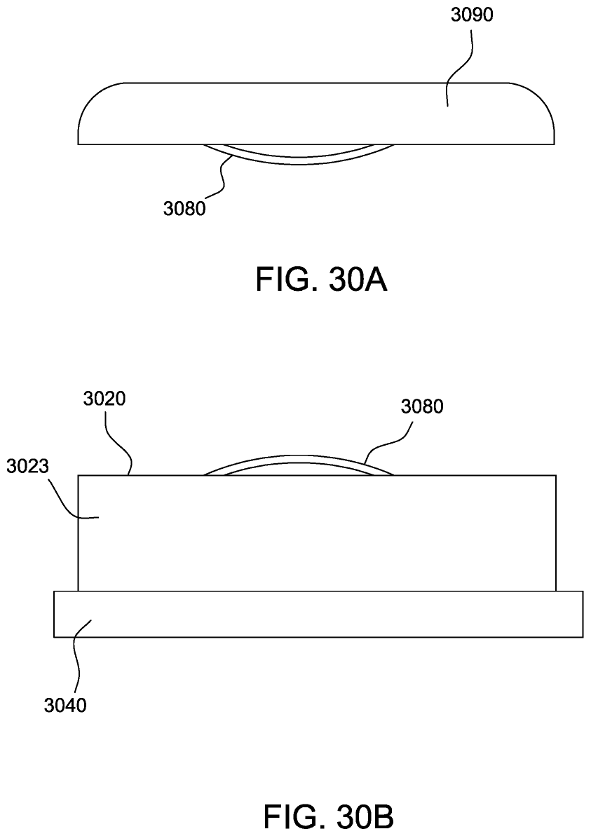

With reference to FIG. 30A, the sensor assembly includes a leaf spring 3080 integrally molded to a bottom surface of the cover 3090. In various aspects, the integrally molded leaf spring 3080 may be of any configuration suitable to prevent the transfer of force to the movable skin sensor 3020 when an external force is exerted on the cover 3090. In various aspects, the leaf spring 3080 may alternatively be molded to a surface of the attachment collar 2310, cover 2390, sliding chassis 2323 and/or frame 2340 (FIG. 23A).

With reference to FIG. 30B, the sensor assembly includes a spring assembly including a leaf spring 3080 integrally molded to one of a top surface of sliding chassis 3023 of the movable skin sensor 3020 or a top surface of frame 3040. In various aspects, the integrally molded spring 3080 may be of any configuration suitable to prevent the transfer of force to the movable skin sensor 3020 when an external force is exerted on the cover 3090. In various aspects, the leaf spring may be manufactured from any suitable material, including plastic materials, metallic materials and the like.

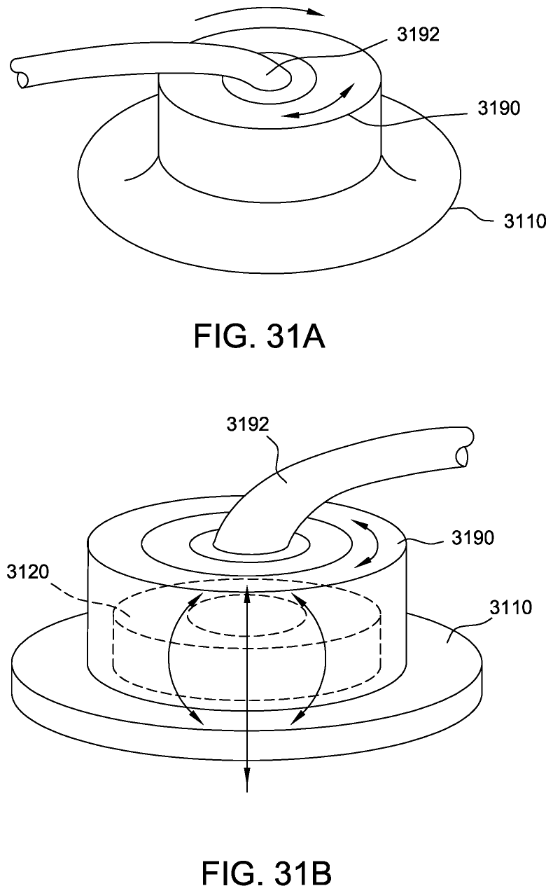

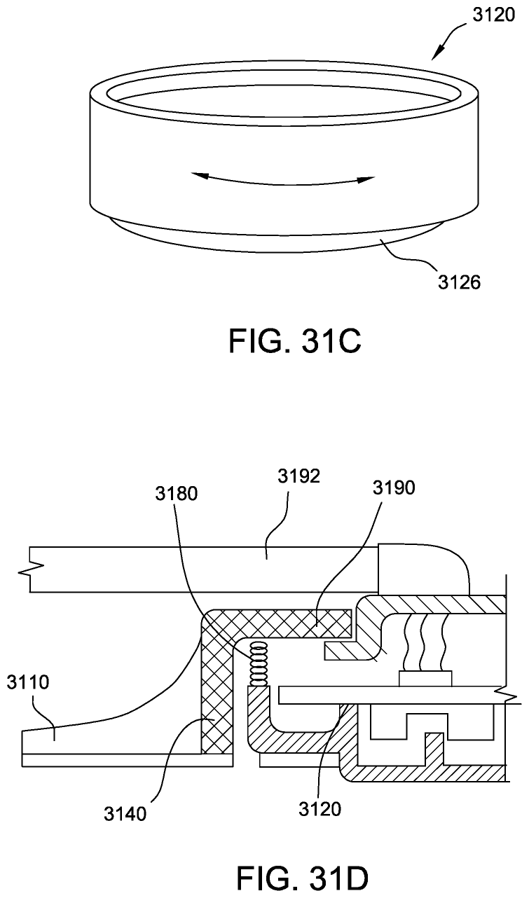

With reference to FIGS. 31A-B, the skin sensor assembly includes a circular cover 3190. A cable 3192 is removably attached to a top surface of the circular cover 3190 and may be configured to rotate independently of the circular cover 3190 in all directions. The circular cover 3190 is configured to be fitted over a circular movable skin sensor 3120 rotatably disposed in an opening of attachment collar 3110. The circular movable skin sensor 3120 may be configured to rotate independently of the circular cover 3190 and/or the attachment collar 3110. The circular movable skin sensor 3120 may further be configured to slide in an upward and downward direction independently of the cover 3190 and/or the circular attachment collar 3110 when an external negative or positive force is exerted on the cover 3190 and/or the circular attachment collar 3110. As shown in more detail in FIG. 31C, the circular movable skin sensor 3120 may additionally include an adhesive layer 3126 configured to more tightly secure the movable skin sensor 3120 to the skin of a patient.

With reference to FIG. 31D, the sensor assembly may include a circular movable skin sensor 3120 configured to rotate independently of frame 3140 with spring 3180 positioned between the movable skin sensor 3120 and the cover 3190. The spring 3180 may be configured to prevent the transfer of force to the movable skin sensor 3120 when an external force is exerted on the cover 3190. In various aspects, the spring 3180 may be configured to exert any level of spring pressure sufficient to prevent the transfer of force exerted on the cover 3190 to the movable skin sensor 3120. In some aspects, the spring 3180 may be configured to exert a light spring pressure on a top surface of the skin sensor 3120. In some aspects, the spring 3180 may be characterized by a low k constant.

Indicator Substances

Suitable indicator substances for use with the methods and devices described herein are disclosed in U.S. 62/577,951, U.S. Pat. Nos. 8,115,000, 8,664,392, 8,697,033, 8,703,100, 8,722,685, 8,778,309, 9,005,581, 9,283,288, 9,376,399, U.S. RE47,413, U.S. RE47,255, U.S. Pat. Nos. 10,137,207, 10,525,149 which are all incorporated by reference in their entirety for all purposes. In some aspects, the indicator substance is any compound eliminated from the body of a patient by glomerular filtration. In some aspects, the indicator substance is any compound that emits fluorescent energy when exposed to electromagnetic radiation and is eliminated from the body of a patient by glomerular filtration. In some aspects, the indicator substance is a GFR agent.

The indicator substance may include but is not limited to acridines, acridones, anthracenes, anthracylines, anthraquinones, azaazulenes, azo azulenes, benzenes, benzimidazoles, benzofurans, benzoindocarbocyanines, benzoindoles, benzothiophenes, carbazoles, coumarins, cyanines, dibenzofurans, dibenzothiophenes, dipyrrolo dyes, flavones, imidazoles, indocarbocyanines, indocyanines, indoles, isoindoles, isoquinolines, naphthacenediones, naphthalenes, naphthoquinones, phenanthrenes, phenanthridines, phenanthridines, phenoselenazines, phenothiazines, phenoxazines, phenylxanthenes, polyfluorobenzenes, purines, pyrazines, pyrazoles, pyridines, pyrimidones, pyrroles, quinolines, quinolones, rhodamines, squaraines, tetracenes, thiophenes, triphenyl methane dyes, xanthenes, xanthones, and derivatives thereof.

The pyrazine may be a compound of Formula I, or a pharmaceutically acceptable salt thereof,

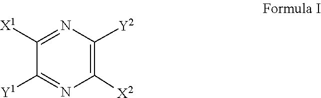

wherein each of X¹ and X² is independently selected from the group consisting of —CN, —CO₂R¹, coNR¹R², CO(AA), —CO(PS) and —CONH(PS); each of Y¹ and Y² is independently selected from the group consisting of —NR¹R² and

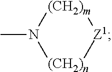

Z¹ is a single bond, —CR′R²—, —O—, —NR¹—, —NCOR¹—, —S—, —SO—, or —SO₂—; each of R¹ to R² are independently selected from the group consisting of H, —CH₂(CHOH)ₐH, —CH₂(CHOH)ₐCH₃, —CH₂(CHOH)ₐCO₂H, —(CHCO₂H)ₐCO₂H, —(CH₂CH₂O)_{c}H, —(CH₂CH₂O)_{c}CH₃, —(CH₂)ₐSO₃H, —(CH₂)ₐSO₃⁻, (CH₂)ₐSO₂H, —(CH₂)ₐSO₂⁻, —(CH₂)ₐNHSO₃H, —(CH₂)ₐNHSO₃⁻, —(CH₂)ₐNHSO₂H, —(CH₂)ₐNHSO₂⁻,—(CH₂)ₐPO₄H₃, —(CH₂)ₐPO₄H₂⁻, —(CH₂)ₐPO₄H²⁻, —(CH₂)ₐPO₄³⁻, —(CH₂)ₐPO₃H₂, —(CH₂)ₐPO₃H⁻, and —(CH₂)ₐPO₃²⁻; (AA) comprises one or more amino acids selected from the group consisting of natural and unnatural amino acids, linked together by peptide or amide bonds and each instance of (AA) may be the same or different than each other instance; (PS) is a sulfated or non-sulfated polysaccharide chain that includes one or more monosaccharide units connected by glycosidic linkages; and ‘a’ is a number from 0 to 10, ‘c’ is a number from 1 to 100, and each of ‘m’ and ‘n’ are independently a number from 1 to 3. In another aspect, ‘a’ is a number from 1 to 10. In still yet another aspect, ‘a’ is 0, 1, 2, 3, 4, 5, 6, 7, 8, 9 or 10.

(AA) comprises one or more natural or unnatural amino acids linked together by peptide or amide bonds. The peptide chain (AA) may be a single amino acid, a homopolypeptide chain or a heteropolypeptide chain, and may be any appropriate length. In some embodiments, the natural or unnatural amino acid is an α-amino acid. In yet another aspect, the α-amino acid is a D-α-amino acid or an L-α-amino acid. In a polypeptide chain that includes two or more amino acids, each amino acid is selected independently of the other(s) in all aspects, including, but not limited to, the structure of the side chain and the stereochemistry. For example, in some embodiments, the peptide chain may include 1 to 100 amino acid(s), 1 to 90 amino acid(s), 1 to 80 amino acid(s), 1 to 70 amino acid(s), 1 to 60 amino acid(s), 1 to 50 amino acid(s), 1 to 40 amino acid(s), 1 to 30 amino acid(s), 1 to 20 amino acid(s), or even 1 to 10 amino acid(s). In some embodiments, the peptide chain may include 1 to 100 α-amino acid(s), 1 to 90 α-amino acid(s), 1 to 80 α-amino acid(s), 1 to 70 α-amino acid(s), 1 to 60 α-amino acid(s), 1 to 50 α-amino acid(s), 1 to 40 α-amino acid(s), 1 to 30 α-amino acid(s), 1 to 20 α-amino acid(s), or even 1 to 10 α-amino acid(s). In some embodiments, the amino acid is selected from the group consisting of D-alanine, D-arginine D-asparagine, D-aspartic acid, D-cysteine, D-glutamic acid, D-glutamine, glycine, D-histidine, D-homoserine, D-isoleucine, D-leucine, D-lysine, D-methionine, D-phenylalanine, D-proline, D-serine, D-threonine, D-tryptophan, D-tyrosine, and D-valine. In some embodiments, the α-amino acids of the peptide chain (AA) are selected from the group consisting of arginine, asparagine, aspartic acid, glutamic acid, glutamine, histidine, homoserine, lysine, and serine. In some embodiments, the α-amino acids of the peptide chain (AA) are selected from the group consisting of aspartic acid, glutamic acid, homoserine and serine. In some embodiments, the peptide chain (AA) refers to a single amino acid (e.g., D-aspartic acid or D-serine).

(PS) is a sulfated or non-sulfated polysaccharide chain including one or more monosaccharide units connected by glycosidic linkages. The polysaccharide chain (PS) may be any appropriate length. For instance, in some embodiments, the polysaccharide chain may include 1 to 100 monosaccharide unit(s), 1 to 90 monosaccharide unit(s), 1 to 80 monosaccharide unit(s), 1 to 70 monosaccharide unit(s), 1 to 60 monosaccharide unit(s), 1 to 50 monosaccharide unit(s), 1 to 40 monosaccharide unit(s), 1 to 30 monosaccharide unit(s), 1 to 20 monosaccharide unit(s), or even 1 to 10 monosaccharide unit(s). In some embodiments, the polysaccharide chain (PS) is a homopolysaccharide chain consisting of either pentose or hexose monosaccharide units. In other embodiments, the polysaccharide chain (PS) is a heteropolysaccharide chain consisting of one or both pentose and hexose monosaccharide units. In some embodiments, the monosaccharide units of the polysaccharide chain (PS) are selected from the group consisting of glucose, fructose, mannose, xylose and ribose. In some embodiments, the polysaccharide chain (PS) refers to a single monosaccharide unit (e.g., either glucose or fructose). In yet another aspect, the polysaccharide chain is an amino sugar where one or more of the hydroxy groups on the sugar has been replaced by an amine group. The connection to the carbonyl group can be either through the amine or a hydroxy group.

Table 1 provides a non-limiting list of exemplary indicator substances. In at least one example, the indicator substance may be 3,6-diamino-2,5-bis{N-[(1R)-1-carboxy-2-hydroxyethyl]carbamoyl}pyrazine. In another example, the indicator substance may be N²,N⁵-bis(2,3-dihydroxypropyl)-3,6-bis[(S)-2,3-dihydroxypropylamino]pyrazine-2,5-dicarboxamide. In another example, the indicator substance may be 3,6-diamino-N²,N⁵-bis((2R,3S,4S,5S)-2,3,4,5,6-pentahydroxyhexyl)pyrazine-2-5-dicarboxamide. In another example, the indicator substance may be 3,6-diamino-N²,N⁵-di(2,5,8,11,14,17,20,23,26,29,32,35,38,41,44,47,50,53,56,59,62,65,68-tricosaoxaheptacontan-70-yl)pyrazine-2,5-dicarboxamide. In yet another example, the indicator substance may be (2R,2′R)-2,2′-((3,6-bis(((S)-2,3-dihydroxypropyl)amino)pyrazine-2,5-dicarbonyl)bis(azanediyl))bis(3-hydroxypropanoic acid). In still another example, the indicator substance may be 3,6-Bis(2,5,8,11,14,17,20,23,26,29,32,35-dodecaoxaheptatriacontan-37-ylamino)-N²,N⁵-di(2,5,8,11,14,17,20,23,26,29,32,35-dodecaoxaheptatriacontan-37-yl)pyrazine-2,5-dicarboxamide. In a further example, the indicator substance may be 3,6-Bis(2,5,8,11,14,17,20,23,26,29,32,35-dodecaoxaoctatriacontan-38-ylamino)-N²,N⁵-di(2,5,8,11,14,17,20,23,26,29,32,35-dodecaoxaheptatriacontan-37-yl)pyrazine-2,5-dicarboxamide. In yet another example, the indicator substance may be D-serine,N,N′-[[3,6-bis[[(2S)-2,3-dihydroxypropyl]amino]-2,5-pyrazinediyl]dicarbonyl]bis-. In an example, the indicator substance may be 3,6-diamino-N²,M-di(2,5,8,11,14,17,20,23,26,29,32,35,38,41,44,47,50,53,56,59,62,65,68,71-tetracosaoxatriheptacontan-73-yl)pyrazine-2,5-dicarboxamide. In another example; the indicator substance may be 3,6-N,N′-Bis(2,3-dihydroxypropyl)-2,5-pyrazinedicarboxamide.

TABLE 1
Indicator Substances

| | Molecular | | |
|---|---|---|---|
| Code | Weight | | |
| Name | (Da) | Structure | Chemical Name |
| MB-102 | 372 | 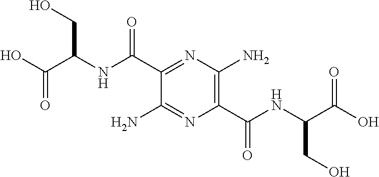 | 3,6-diamino-2,5-bis{N-[(1R)-1- carboxy-2- hydroxyethyl]cathamoyl}pyrazine |
| MB-404 | 492 | 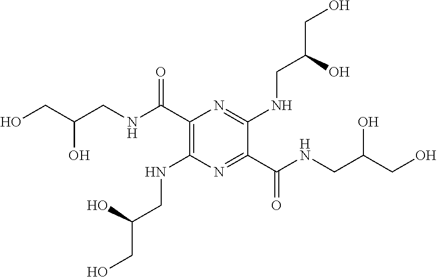 [SMILES: O=C(NCC(O)CO)c1nc(NC[C@H](O)CO)c(C(=O)NCC(O)CO)nc1NC[C@H](O)CO] | N²,N⁵-bis(2,3-dihydroxypropyl)- 3,6-bis[(S)-2,3- dihydroxypropylamino]pyrazine- 2,5-dicarboxamide |
| MB-106 | 524 | 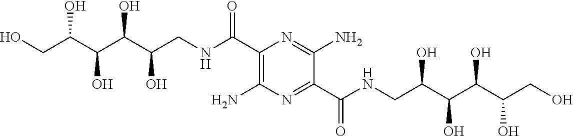 [SMILES: Nc1nc(C(=O)NC[C@@H](O)[C@H](O)[C@@H](O)[C@@H](O)CO)c(N)nc1C(=O)NC[C@@H](O)[C@H](O)[C@@H](O)[C@@H](O)CO] | 3,6-diamino-N²,N⁵- bis((2R,3S,4S,5S)-2,3,4,5,6- pentahydroxyhexyl)pyrazine-2- 5-dicarboxamide |
| MB-216 | 2367 | 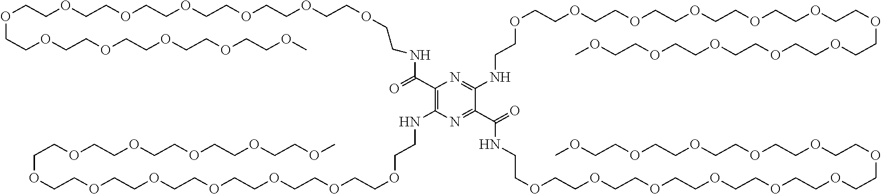 [SMILES: COCCOCCOCCOCCOCCOCCOCCOCCOCCOCCOCCOCCNC(=O)c1nc(NCCOCCOCCOCCOCCOCCOCCOCCOCCOCCOCCOCCOC)c(C(=O)NCCOCCOCCOCCOCCOCCOCCOCCOCCOCCOCCOCCOC)nc1NCCOCCOCCOCCOCCOCCOCCOCCOCCOCCOCCOCCOC] | 3,6- Bis(2,5,8,11,14,17,20,23,26,29,32, 35-dodecaoxaheptatriacontan- 37-ylamino)-N²,N⁵- di(2,5,8,11,14,17,20,23,26,29,32, 35-dodecaoxaheptatriacontan- 37-yl)pyrazine- 2,5-dicarboxamide |
| MB-212 | 2395 | 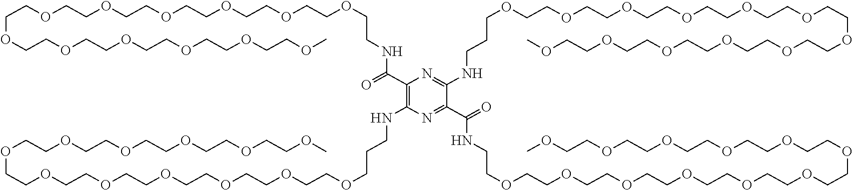 [SMILES: COCCOCCOCCOCCOCCOCCOCCOCCOCCOCCOCCOCCCNc1nc(C(=O)NCCOCCOCCOCCOCCOCCOCCOCCOCCOCCOCCOCCOC)c(NCCCOCCOCCOCCOCCOCCOCCOCCOCCOCCOCCOCCOC)nc1C(=O)NCCOCCOCCOCCOCCOCCOCCOCCOCCOCCOCCOCCOC] | 3,6-Bis(2,5,8,11,14,17,20,23,26,29,32, 35-dodecaoxaoctatriacontan- 38-ylamino)-N²,N⁵- di(2,5,8,11,14,17,20,23,26,29,32, 35-dodecaoxaheptatriacontan- 37-yl)pyrazine-2,5- dicarboxamide |
| MB-116 | 2250 | 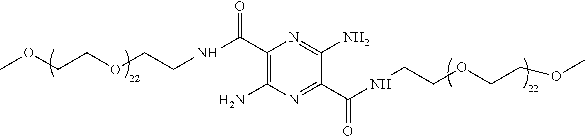 | 3,6-diamino-N²,N⁵- di(2,5,8,11,14,17,20,23,26,29,32, 35,38,41,44,47,50,53,56,59,62,65, 68-tricosaoxaheptacontan-70- yl)pyrazine-2,5-dicarboxamide |
| MB-206 | 520 | 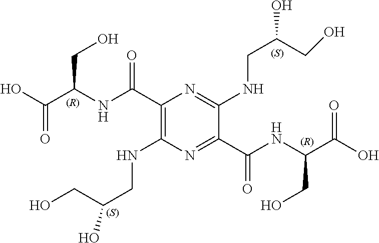 | D-Serine,N,N′-[[3,6-bis[[(2S)- 2,3-dihydroxypropyl]amino]- 2,5-pyrazinediyl]dicarbonyl]bis- |
| MB-112 | 2339 | 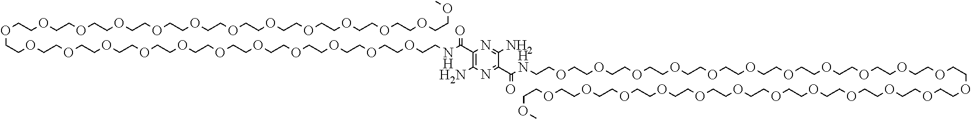 [SMILES: COCCOCCOCCOCCOCCOCCOCCOCCOCCOCCOCCOCCOCCOCCOCCOCCOCCOCCOCCOCCOCCOCCOCCOCCNC(=O)c1nc(N)c(C(=O)NCCOCCOCCOCCOCCOCCOCCOCCOCCOCCOCCOCCOCCOCCOCCOCCOCCOCCOCCOCCOCCOCCOCCOCCOC)nc1N] | 3,6-diamino-N²,N⁵- di(2,5,8,11,14,17,20,23,26,29,32, 35,38,41,44,47,50,53,56,59,62,65, 68,71- tetracosaoxatriheptacontan-73- yl)pyrazine-2,5-dicarboxamide |
| MB-402 | 344 | 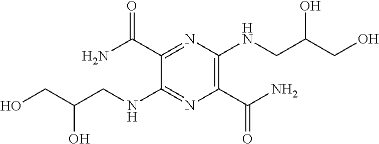 [SMILES: NC(=O)c1nc(NCC(O)CO)c(C(N)=O)nc1NCC(O)CO] | 3,6-N,N′-Bis(2,3- dihydroxypropyl)-2,5- pyrazinedicarboxamide |

Other examples of indicator substances include, but are not limited to, 3,6-diamino-N²,N²,N⁵,N⁵-tetrakis(2-methoxyethyl)pyrazine-2,5-dicarboxamide, 3,6-diamino-N²,N⁵-bis(2,3-dihydroxypropyl)pyrazine-2,5-dicarboxamide, (2S,2'S)-2,2′-((3,6-diaminopyrazine-2,5-dicarbonyl)bis(azanediyl))bis(3-hydroxypropanoic acid), 3,6-bis(bis(2-methoxyethyl)amino)-N²,N²,N⁵,N⁵-tetrakis(2-methoxyethyl) pyrazine-2,5-dicarboxamide bis(TFA) salt, 3,6-diamino-N²,N⁵-bis(2-aminoethyl)pyrazine-2,5-dicarboxamide bis(TFA) salt, 3,6-diamino-N²,N⁵-bis (D-aspartate)-pyrazine-2,5-dicarboxamide, 3,6-diamino-N²,N⁵-bis(14-oxo-2,5,8,11-tetraoxa-15-azaheptadecan-17-yl)pyrazine-2,5-dicarboxamide, 3,6-diamino-N²,N⁵-bis(26-oxo-2,5,8,11,14,17,20,23-octaoxa-27-azanonacosan-29-yl)pyrazine-2,5-dicarboxamide, 3,6-diamino-N²,N⁵-bis(38-oxo-2,5,8,11,14,17,20,23,26,29,32,35-dodecaoxa-39-azahentetracontan-41-yl)pyrazine-2,5-dicarboxamide, bis(2-(PEG-5000)ethyl) 6-(2-(3,6-diamino-5-(2-aminoethylcarbamoyl) pyrazine-2-carboxamido)ethylamino)-6-oxohexane-1,5-diyldicarbamate, (R)-2-(6-(bis(2-methoxyethyl)amino)-5-cyano-3-morpholinopyrazine-2-carboxamido)succinic acid, (2R,2′R)-2,2′-((3,6-diaminopyrazine-2,5-dicarbonyl)bis(azanediyl))bis(3-hydroxypropanoic acid), (2S,2'S)-2,2′-((3,6-diaminopyrazine-2,5-dicarbonyl)bis(azanediyl))bis(3-hydroxypropanoic acid), (2R,2′R)-2,2′-((3,6-diaminopyrazine-2,5-dicarbonyl)bis(azanediyl)) dipropionic acid, 3,3′-((3,6-diaminopyrazine-2,5-dicarbonyl)bis(azanediyl))dipropionic acid, 2,2′4-(3,6-diaminopyrazine-2,5-dicarbonyl)bis(azanediyl))diacetic acid, (2S,2'S)-2,2′-((3,6-diaminopyrazine-2,5-dicarbonyl)bis(azanediyl)) dipropionic acid, 2,2′-((3,6-diaminopyrazine-2,5-dicarbonyl)bis(azanediyl))bis(2-methylpropanoic acid), 3,6-diamino-N²,N⁵-bis((1R,2S,3R,4R)-1,2,3,4,5-pentahydroxypentyl) pyrazine-2,5-dicarboxamide. In some aspects, the indicator substance is (2R,2′R)-2,2′-((3,6-diaminopyrazine-2,5-dicarbonyl)bis(azanediyl))bis(3-hydroxypropanoic acid). In some aspects, the indicator substance is (2S,2'S)-2,2′-((3,6-diaminopyrazine-2,5-dicarbonyl)bis(azanediyl))bis(3-hydroxypropanoic acid).

In some aspects, the indicator substance is (2R,2′R)-2,2′-((3,6-diamino-pyrazine-2,5-dicarbonyl)bis(azanediyl))bis(3-hydroxypropanoic acid) (also known as 3,6-diamino-N2,N5-bis(D-serine)-pyrazine-2,5-dicarboxamide),

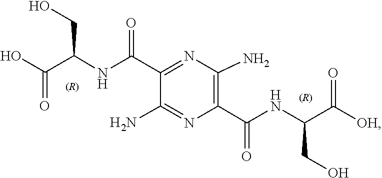

or a pharmaceutically acceptable salt thereof.

In some aspects, the indicator substance is (2S,2′S)-2,2′-((3,6-diamino-pyrazine-2,5-dicarbonyl)bis(azanediyl))bis(3-hydroxypropanoic acid) (also known as 3,6-diamino-N2,N5-bis(L-serine)-pyrazine-2,5-dicarboxamide),

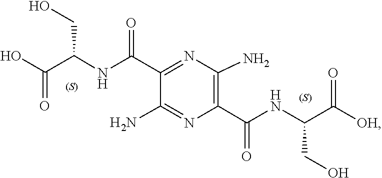

or a pharmaceutically acceptable salt thereof.

In any aspect of the indicator substance, one or more atoms may alternatively be substituted with an isotopically labelled atom of the same element. For example, a hydrogen atom may be isotopically labelled with deuterium or tritium; a carbon atom may be isotopically labelled with ¹³C or ¹⁴C; a nitrogen atom may be isotopically labelled with ¹⁴N or ¹⁵N. An isotopic label may be a stable isotope or may be an unstable isotope (i.e., radioactive). The indicator substance may contain one or more isotopic labels. The isotopic label may be partial or complete. For example, an indicator substance may be labeled with 50% deuterium thereby giving the molecule a signature that can be readily monitored by mass spectroscopy or other technique. As another example, the indicator substance may be labeled with tritium thereby giving the molecule a radioactive signature that can be monitored both in vivo and ex vivo using techniques known in the art.

Pharmaceutically acceptable salts are known in the art. In any aspect herein, the indicator substance may be in the form of a pharmaceutically acceptable salt. By way of example and not limitation, pharmaceutically acceptable salts include those as described by Berge, et al. in J. Pharm. Sci., 66(1), 1 (1977), which is incorporated by reference in its entirety for all purposes. The salt may be cationic or anionic. In some embodiments, the counter ion for the pharmaceutically acceptable salt is selected from the group consisting of acetate, benzenesulfonate, benzoate, besylate, bicarbonate, bitartrate, bromide, calcium edetate, camsylate, carbonate, chloride, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, methylsulfate, mucate, napsylate, nitrate, pamoate, pantothenate, phosphate, diphosphate, polygalacturonate, salicylate, stearate, subacetate, succinate, sulfate, tannate, tartrate, teoclate, triethiodide, adipate, alginate, aminosalicylate, anhydromethylenecitrate, arecoline, aspartate, bisulfate, butylbromide, camphorate, digluconate, dihydrobromide, disuccinate, glycerophosphate, jemisulfate, judrofluoride, judroiodide, methylenebis(salicylate), napadisylate, oxalate, pectinate, persulfate, phenylethylbarbarbiturate, picrate, propionate, thiocyanate, tosylate, undecanoate, benzathine, chloroprocaine, choline, diethanolamine, ethylenediamine, meglumine, procaine, benethamine, clemizole, diethylamine, piperazine, tromethamine, aluminum, calcium, lithium, magnesium, potassium, sodium zinc, barium and bismuth. Any functional group in the indicator substance capable of forming a salt may optionally form one using methods known in the art. By way of example and not limitation, amine hydrochloride salts may be formed by the addition of hydrochloric acid to the indicator substance. Phosphate salts may be formed by the addition of a phosphate buffer to the indicator substance. Any acid functionality present, such as a sulfonic acid, a carboxylic acid, or a phosphonic acid, may be deprotonated with a suitable base and a salt formed. Alternatively, an amine group may be protonated with an appropriate acid to form the amine salt. The salt form may be singly charged, doubly charged or even triply charged, and when more than one counter ion is present, each counter ion may be the same or different than each of the others.

Methods of Use

Suitable methods of use for the devices described herein are disclosed in U.S. Pat. Nos. 8,115,000, 9,283,288, 9,632,094, 10,194,854, 10,548,521, 10,525,149, US 20150147277, US 20190125901, US 20190125902, U.S. Ser. No. 16/552,474, U.S. Ser. No. 16/552,609, and U.S. 63/169,568, which are all incorporated by reference in their entirety for all purposes.

In some aspects, disclosed herein is a method for assessing the physiological function of body cells of a patient. The method generally comprises: applying a sensor assembly onto the body surface of the patient, administering into the body of the patient an indicator substance, said indicator substance configured to generate an optical response in response to an interrogation light; detecting said optical response using the sensor assembly over a predetermined period of time; and determining the physiological function of body cells in said patient based on the detected optical response.

In yet another aspect, disclosed herein is a method for determining a glomerular filtration rate (GFR) in a patient in need thereof. The method generally comprises: applying a sensor assembly onto the body surface of the patient, administering into the body of the patient an indicator substance, said indicator substance configured to generate an optical response in response to an interrogation light; detecting said optical response using the sensor assembly over a predetermined period of time; and determining the GFR in said patient based on the detected optical response.

In some aspects of the method for determining the GFR in a patient, the sensor assembly is as described elsewhere herein. In some aspects of the method for determining the GFR in a patient, the indicator substance is as described elsewhere herein. In some aspects of the method for determining the GFR in a patient, the indicator substance is (2R,2′R)-2,2′-((3,6-diamino-pyrazine-2,5-dicarbonyl)bis-(azanediyl))bis(3-hydroxypropanoic acid) (also known as MB-102 or 3,6-diamino-N2,N5-bis(D-serine)-pyrazine-2,5-dicarboxamide),

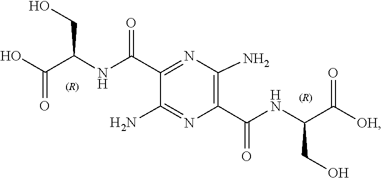

or a pharmaceutically acceptable salt thereof.

In still yet another aspect, disclosed herein is a method for assessing gut function in a subject in need thereof. The method generally comprises applying a sensor assembly onto the body surface of the patient, administering an effective amount of a composition for assessing gut function comprising an indicator substance, irradiating the composition absorbed by the subject's gut with non-ionizing radiation from the sensor assembly, wherein the radiation causes the composition to fluoresce; transcutaneously detecting the fluorescence of the indicator substance using the sensor assembly; and assessing gut function in the subject based on the detected fluorescence.

In still yet another aspect, disclosed herein is a method of monitoring mucosal healing in a patient with a digestive disease or in a pre-disease state. The method generally comprises establishing a baseline of the patient, treating the patient for the digestive disease or the pre-disease state, measuring transdermally gut permeability of the patient after treatment via a sensor assembly, and comparing a second total percentage of the administered dose recovered to a baseline total percentage of the administered dose recovered. Establishing the baseline of the patient may include enterally administering a first dosage of a composition comprising a an indicator substance that is not substantially absorbed by a healthy gut, measuring, via the sensor assembly, a first amount of the administered dose that can be found outside the gut over a period of time, and determining the baseline total percentage of the administered dose recovered. Measuring gut permeability of the patient after treatment may further comprise enterally administering a second dosage of the composition comprising the indicator substance, measuring, via the sensor assembly, a second amount of the enterally administered dose that can be found outside the gut over a period of time, and determining the second total percentage of the administered dose recovered.

In other aspects, disclosed herein is a method for determining mucosal healing in a patient with a digestive disease. The method generally comprises enterally administering a dosage of a composition comprising an indicator substance that is not substantially absorbed by a healthy gut, measuring transdermally, via a sensor assembly, an amount of the administered dose that can be found outside the gut over a period of time, and determining a total percentage of the administered dose recovered. In some aspects, the total percentage of the administered dose recovered correlates to the patient's mucosal healing.

In still yet another aspect, disclosed herein is a method for determining a dosing prescription for a medicament. The method generally comprises applying a sensor assembly onto the body surface of the patient, administering to the bloodstream of the patient an indicator substance; administering to the patient at least one dose of a medicament wherein administering the indicator substance and the medicament to the patient is either sequential or simultaneous; exposing the indicator substance to visible or infrared light, thereby causing spectral energy to emanate from the indicator substance; monitoring transcutaneously a change in the spectral energy from the indicator substance over a period of time using the sensor assembly; correlating a change in intensity of the spectral energy from the indicator substance to a clearance rate of the indicator substance from the bloodstream in the patient; calculating a clearance rate of the medicament in the patient based on the clearance rate of the indicator substance; determining an amount of the medicament in the bloodstream of the patient as a function of time based on the clearance rate of the medicament; and adjusting the dosing prescription of the medicament to the patient based on the amount of the medicament in the bloodstream of the patient as a function of time thereby determining the dosing prescription for the medicament to the patient.

This written description uses examples to disclose the subject matter herein, including the best mode, and also to enable any person skilled in the art to practice the subject matter disclosed herein, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A sensor assembly comprising:
a housing assembly configured to attach to a body surface of a patient and comprising at least one opening;
a skin sensor movably disposed in the at least one opening of the housing assembly; and
wherein the housing assembly is configured to move independently relative to the skin sensor when an external force is applied to the housing assembly.

2. The sensor assembly according to claim 1, wherein the housing assembly comprises an attachment collar and a cover attached to the attachment collar.

3. The sensor assembly according to claim 2, wherein the attachment collar comprises at least one opening and wherein the skin sensor is movably disposed in the at least one opening of the attachment collar.

4. The sensor assembly according to claim 1, wherein the skin sensor is slidably disposed in the at least one opening of the housing assembly.

5. The sensor assembly according to claim 1, wherein the skin sensor is rotatably disposed in the at least one opening of the housing assembly.

6. The sensor assembly according to claim 1, wherein the housing assembly further comprises a frame positioned in the at least one opening of the housing assembly and configured to slidably receive the skin sensor in the at least one opening of the housing assembly.

7. The sensor assembly according to claim 1, wherein the external force is a positive, negative or rotational force applied to the outer surface of the housing assembly.

8. The sensor assembly according to claim 1, wherein the sensor assembly further comprises one or more biasing assemblies configured to prevent a transfer of externally applied force to the skin sensor.

9. The sensor assembly according to claim 2, wherein the sensor assembly further comprises one or more biasing assemblies configured to prevent a transfer of externally applied force to the skin sensor.

10. The sensor assembly according to claim 1, wherein the skin sensor is configured to be tiltable in at least one direction relative to the at least one opening of the housing assembly.

11. The sensor assembly according to claim 10, wherein the skin sensor is configured to be tiltable in all directions relative to the at least one opening of the housing assembly.

12. The sensor assembly according to claim 6, wherein the frame comprises a plurality of ribs arranged around an inner perimeter of the frame and the skin sensor comprises a plurality of slots arranged around an outer perimeter of the skin sensor and wherein the plurality of ribs arranged around an inner perimeter of the frame are configured to slidably mate with the plurality of slots arranged around an outer perimeter of the skin sensor.

13. The sensor assembly according to claim 3, wherein the skin sensor is configured to extend beyond the at least one opening of the attachment collar to form a protrusion.

14. The sensor assembly according to claim 1, wherein the sensor assembly further comprises a controller.

15. A sensor assembly comprising:
an attachment collar configured to attach to a body surface of a patient and comprising at least one opening;
a skin sensor movably disposed in the at least one opening of the attachment collar; and
a cover attached to the attachment collar;
wherein the attachment collar and cover are configured to move independently relative to the skin sensor when an external force is applied to the sensor assembly.

16. The sensor assembly according to claim 15, wherein the skin sensor is configured to extend beyond the attachment collar to form a protrusion.

17. The sensor assembly according to claim 16, wherein the skin sensor further comprises an adhesive layer on at least a portion of a bottom surface of the skin sensor surrounding the protrusion, and wherein the attachment collar further comprises an adhesive layer on at least a portion of a bottom surface of the attachment collar.

18. A sensor assembly comprising:
an attachment collar configured to attach to a body surface of a patient and comprising at least one opening,
a skin sensor disposed in the at least one opening of the attachment collar, and
a cover attached to the attachment collar and at least partially enclosing the skin sensor,
wherein the cover and the attachment collar are configured to rotate independently of the skin sensor when a rotational force is applied to the sensor assembly.

19. The sensor assembly of claim 18, wherein the sensor assembly further comprises a cable rotatably secured to a top surface of the cover.

20. The sensor assembly of claim 19, wherein the cable is configured to rotate independently of the skin sensor when a rotational force is applied to the cable.
